(19) United States
(12) Patent Application Publication
(10) Pub. No.: US 2026/0284663 A1
(43) Pub. Date: Sep. 24, 2026

(54) SYSTEMS AND METHODS FOR GENERATING DROPLETS AND PERFORMING DIGITAL ANALYSES

(71) Applicant: Countable Labs, Inc., Palo Alto, CA (US)

(72) Inventors: Hei Mun Christina FAN, Palo Alto, CA (US); Janice Hoiyi LAI, Mountain View, CA (US); Sixing LI, Mountain View, CA (US); Stephen P.A. FODOR, Palo Alto, CA (US); Eleen Yee Lam SHUM, San Carlos, CA (US)

(21) Appl. No.: 19/701,621

(22) Filed: Jun. 8, 2026

Related U.S. Application Data
(63) Continuation-in-part of application No. 19/648,997, filed on Apr. 15, 2026.
Application No. 19/648,997 is a continuation of application No. 19/400,892, filed on Nov. 25, 2025, now Pat. No. 12,631,651.
Application No. 19/400,892 is a continuation of application No. 19/063,118, filed on Feb. 25, 2025, now Pat. No. 12,540,948.
Application No. 19/063,118 is a continuation of application No. 18/948,248, filed on Nov. 14, 2024, now Pat. No. 12,270,815.
Application No. 18/948,248 is a continuation of application No. 18/646,572, filed on Apr. 25, 2024, now Pat. No. 12,265,088.
Application No. 18/646,572 is a continuation of application No. 17/711,417, filed on Apr. 1, 2022, now Pat. No. 12,000,842.
Application No. 17/711,417 is a continuation of application No. PCT/US2022/018994, filed on Mar. 4, 2022.
(60) Provisional application No. 63/157,292, filed on Mar. 5, 2021.

(51) Int. Cl. B01L 3/00; C12Q 1/6844; G01N 21/64; G01N 33/68

(52) U.S. Cl. CPC B01L 3/502753; B01L 3/502715; C12Q 1/6844; G01N 21/645; G01N 21/6486; G01N 33/689; B01L 2200/027; B01L 2200/0689; B01L 2300/0681; B01L 2400/0409; G01N 2021/6482

(57) ABSTRACT

The disclosure provides compositions, methods, and systems for implementation of high-performance molecular diagnostic assays, where generation of counts of targets from a sample is achieved in a rapid manner, with respect to sample partitioning and target detection, in the single-molecule regime.

### CROSS-REFERENCE

This application is a continuation in part application of U.S. patent application Ser. No. 19/648,997, filed Apr. 15, 2026, which is a continuation of U.S. patent application Ser. No. 19/400,892, filed Nov. 25, 2025, now U.S. Pat. No. 12,631,651, which is a continuation of U.S. patent application Ser. No. 19/063,118, filed Feb. 25, 2025, now U.S. Pat. No. 12,540,948, which is a continuation application of U.S. patent application Ser. No. 18/948,248, filed Nov. 14, 2024, now U.S. Pat. No. 12,270,815, issued Apr. 8, 2025, which is a continuation application of U.S. patent application Ser. No. 18/646,572, filed Apr. 25, 2024, now U.S. Pat. No. 12,265,088, issued Apr. 1, 2025, which is a continuation application of U.S. patent application Ser. No. 17/711,417, filed Apr. 1, 2022, now U.S. Pat. No. 12,000,842, issued Jun. 4, 2024, which is a continuation of PCT Patent Application number PCT/US 2022/018994, filed Mar. 4, 2022, which claims the benefit of U.S. Provisional Application number 63/157,292, filed Mar. 5, 2021, each of which is incorporated in its entirety herein by this reference.

### TECHNICAL FIELD

The disclosure generally relates to systems, methods, and compositions for high dynamic range digital counting of targets.

### BACKGROUND

Unambiguous detection and counting of sample targets has utility in many fields. In particular, the ability to count, with high accuracy, across a high dynamic range of potential target numbers can contribute to generation of diagnoses and/or support interpretation of assay results, which can be used to provide healthcare or otherwise improve outcomes in other fields of interest. A single platform that has the ability to accurately count across a high dynamic range could theoretically provide value in relation to rare molecule detection and to also provide value for assays requiring higher count levels.

High dynamic range digital quantitation of targets using partition-based systems (e.g., as in digital PCR) has, however, been traditionally limited by the number of partitions available, partition format (e.g., providing only 1D or 2D formats), high apparatus costs, operation in a high-occupancy regime requiring statistical error correction factors for assessment of results, material costs, insufficient precision, insufficient accuracy, low speed performance, inability to preserve the sample for further analysis, and other factors. Such traditional approaches include methods to compensate for deficiencies (e.g., with use of computational tools that ultimately reduce counting accuracy and/or dynamic range). Limitations of existing approaches can however, be overcome by the invention(s) described in following sections.

As such, there is a need for innovation in fields related to systems, compositions, and methods for multiplexed detection and quantitation of targets.

### SUMMARY OF THE INVENTION

Currently, platforms, methods, and compositions for target quantification (e.g., counting, enumeration) involve significant investments in platform cost and computational resource use. Even then, such technologies are limited in relation to: high reagent use requirements, high dead volume associated with sample waste, and physical limitations in relation to the number of useable partitions available for target capture and subsequent detection.

Further limitations of other platforms, methods, and compositions for target quantification include limitations in available partition format (e.g., providing only 1D or 2D formats), high apparatus costs, operation in a high-occupancy regime requiring statistical error correction factors for assessment of results, material cost, insufficient precision, insufficient accuracy, low speed performance, inability to preserve the sample for further analysis, and other factors.

Accordingly, this disclosure describes embodiments, variations, and examples of systems, methods, and compositions for high dynamic range digital detection of a large number of targets, in a high-performance manner, and with less complex instrumentation.

An aspect of the disclosure provides systems, methods, and compositions for counting or otherwise detecting presence of targets distributed across partitions, where counting of targets of a sample is achievable across a dynamic range of five logarithms, across a dynamic range of six logarithms, across a dynamic range of seven logarithms, across a dynamic range of eight logarithms, or greater. In embodiments, systems, methods, and compositions described can enable precise and accurate counting of more than one target of a sample, more than 100 targets of a sample, more than 1,000 targets of a sample, more than 10,000 targets of a sample, more than 100,000 targets of a sample, more than 1,000,000 targets of a sample, more than 10,000,000 targets of a sample, or more than 100,000,000 targets of a sample, where the targets are individually distributed across partitions, such that no partition contains more than one target (e.g., each partition has zero or one target).

An aspect of the disclosure provides systems, methods, and compositions for counting or otherwise detecting presence of targets distributed across partitions, where a generated count has an accuracy greater than 90%, an accuracy greater than 91%, an accuracy greater than 92%, an accuracy greater than 93%, an accuracy greater than 94%, an accuracy greater than 95%, an accuracy greater than 96%, an accuracy greater than 97%, an accuracy greater than 98%, an accuracy greater than 99%, an accuracy greater than 99.5%, an accuracy greater than 99.9%, or greater. Accuracy can be determined based upon comparing determined counts and expected or known counts for a sample (e.g., for a test sample where the number of targets is known prior to processing the sample according to aspects of the inventions described).

An aspect of the disclosure provides systems, methods, and compositions for counting or otherwise detecting presence of targets distributed across partitions, where a generated count has a precision characterized by a coefficient of variation less than 10%, characterized by a coefficient of variation less than 9%, characterized by a coefficient of variation less than 8%, characterized by a coefficient of variation less than 7%, characterized by a coefficient of variation less than 6%, characterized by a coefficient of variation less than 5%, characterized by a coefficient of variation less than 4%, characterized by a coefficient of variation less than 3%, characterized by a coefficient of variation less than 2%, characterized by a coefficient of variation less than 1%, or better. An aspect of the disclosure provides systems, methods, and compositions for counting or otherwise detecting presence of targets distributed across partitions, where a generated count has a precision characterized by an uncertainty proportional to the square root of the number of targets, such that the coefficient of variation may be greater than 10% (e.g., less than 50%, less than 40%, less than 30%, less than 20%, etc.) in the rare molecule regime. In variations, precision can be characterized in relation to another measure of statistical variability.

In relation to generating the count, detection of signal-positive partitions containing respective targets of the sample can involve a 3D imaging technique (e.g., light sheet imaging, 3D confocal microscopy, etc.) or a non-3D imaging technique. In embodiments, generating the count can include scanning a set of cross sections of a closed container containing a set of partitions, each having zero or one target of the sample. In some embodiments, one or more partitions can have more than one target. In embodiments, readout can be performed for each of a set of cross sections of the plurality of partitions/closed container, across multiple color channels (e.g., 2 color channels, three color channels, four color channels, five color channels, six color channels, seven color channels, etc.).

In relation to droplet/emulsion digital PCR, the invention(s) enable readout of one or more cross-sections of a closed collecting container to generate counts described, where, for each channel, readout can be performed within a duration of 5 minutes, 4 minutes, 3 minutes, 2 minutes, 1 minute, 30 seconds, 20 seconds, 10 seconds, or less.

The invention(s) also enable generating counts (e.g., counts per channel, counts across sets of channels) from readout data with high performance, where transforming readout data into counts can be performed within 120 seconds, within 100 seconds, within 90 seconds, within 80 seconds, within 70 seconds, within 60 seconds, within 50 seconds, within 40 seconds, within 30 seconds, 20 seconds, within 15 seconds, within 10 seconds, within 9 seconds, within 8 seconds, within 7 seconds, within 6 seconds, within 5 seconds, within 4 seconds, within 3 seconds, within 2 seconds, within 1 second, within 0.5 seconds, within 0.1 seconds, or less.

In embodiments, the disclosure provides systems, methods, and compositions for generating, from a sample, an emulsion having a set of droplets (e.g., as a dispersed phase), within a continuous phase, wherein each of the set of droplets contains zero or one target of the sample (i.e., no droplet of the set of droplets contains two or more targets of the sample). In embodiments, droplets of the set of droplets are aqueous (e.g., derived from the sample), and are each surrounded by a thin film of liquid that is immiscible with the aqueous droplets. The continuous phase of the emulsion is also aqueous.

In embodiments, generation of the emulsion according to methods described produces a clear emulsion. In one variation, clarity can be characterized in relation to transmissivity as detectable by a transmission detector and/or in relation to a suitable distance or depth (e.g., depth or distance into a collecting container for the emulsion; through a depth of a container of the emulsion, along an axis in which measurement of clarity is performed, etc.), where, in variations, the threshold level of clarity of the emulsion is associated with a transmissivity greater than 70% transmissivity, greater than 80% transmissivity, greater than 90% transmissivity, greater than 95% transmissivity, greater than 99% transmissivity, etc. As such, in accordance with methods described, upon measuring clarity of the emulsion (e.g., later stage emulsion) using a transmission detector the emulsion is characterized by a clarity associated with greater than 70% transmissivity, greater than 80% transmissivity, greater than 90% transmissivity, greater than 95% transmissivity, greater than 99% transmissivity, etc., which is above the threshold level of clarity.

In embodiments, generation of the emulsion according to methods described produces an emulsion having gel-like properties. In embodiments, the emulsion having gel-like properties cannot or does not flow (e.g., through microfluidic structures) upon generation. In embodiments, generation of the emulsion produces droplets of the emulsion within a closed collecting container, where the closed collecting container remains closed throughout optical interrogation of the emulsion for detection of targets. In embodiments, the emulsion is stabilized in position within the closed collecting container upon generation of the emulsion, such that droplets of the emulsion do not move relative to each other and/or relative to the closed collecting container upon generation and stabilization in position within the closed collecting container. Such gel-like properties enables repeat scanning of each droplet using different interrogation channels, where a compiled optical signature can be aggregated from optical signatures transmitted from each droplet, where individual optical signatures are generated using each of a set of interrogation channels. Each of the set of interrogation channels can have its own excitation profile and emission profile.

Gel-like aspects of the emulsion can be characterized in terms of shear-thickening/dilatancy behavior, viscosity, modulus, or other mechanical parameters. In embodiments, a gel-like emulsion generated according to methods described can have a viscosity of greater than 100 centipoise, greater than 1000 centipoise, greater than 10,000 centipoise, greater than 50,000 centipoise, greater than 100,000 centipoise, or greater.

Performance values described are achieved in part due to the high number of partitions involved when using the systems, methods, and compositions described, where distribution of sample targets across partitions results in low occupancy of partitions by targets, and large partition numbers contribute to significantly low or non-existent percentages of doublets (e.g., single partitions occupied by two targets), triplets (e.g., single partitions occupied by three targets), or other forms of multi-plets (single partitions occupied by multiple targets). In particular, successful characterization across such a wide dynamic range is attributed to the high degree of partitioning (with achievable numbers of generated partitions described) and extremely low occupancy (with achievable percent occupancies described), such that multiple molecules from the target molecules of interest have a minimal (or zero) probability of occupying the same partition as another target molecule. In such a high-partition and low-occupancy regime, there is no competition associated with multiple target molecules per partition, and the platform is not subject to problems related to differences in PCR efficiency between different target molecules.

Examples of partition generation methods can include generating an extremely high number of droplets (e.g., greater than 5 million droplets, greater than 6 million droplets, greater than 7 million droplets, greater than 8 million droplets, greater than 9 million droplets, greater than 10 million droplets, greater than 15 million droplets, greater than 20 million droplets, greater than 25 million droplets, greater than 30 million droplets, greater than 40 million droplets, greater than 50 million droplets, greater than 100 million droplets, greater than 1 billion droplets, etc.) within a collecting container having a volumetric capacity (e.g., less than 50 microliters, from 50 through 100 microliters and greater, etc.), where droplets have a characteristic dimension (e.g., from 1-50 micrometers, from 10-50 micrometers, etc.) that is relevant for digital analyses, target detection, individual molecule partitioning, or other applications.

In relation to occupancy, embodiments, variations, and examples of partitioning are conducted in a manner such that each partition has one or zero molecules, such that the partitions are characterized as having low occupancy (e.g., less than 15% occupancy of partitions by individual molecules, less than 14% occupancy of partitions by individual molecules, less than 13% occupancy of partitions by individual molecules, less than 12% occupancy of partitions by individual molecules, less than 11% occupancy of partitions by individual molecules, less than 10% occupancy of partitions by individual molecules, less than 9% occupancy of partitions by individual molecules, less than 8% occupancy of partitions by individual molecules, less than 7% occupancy of partitions by individual molecules, less than 6% occupancy of partitions by individual molecules, less than 5% occupancy of partitions by individual molecules, less than 4% occupancy of partitions by individual molecules, etc.).

In examples, the systems, methods, and compositions described can be used to generate 50,000 counts per target for each of a set of targets of interest, 60,000 counts per target for each of a set of targets of interest, 70,000 counts per target for each of a set of targets of interest, 80,000 counts per target for each of a set of targets of interest, 90,000 counts per target for each of a set of targets of interest, 100,000 counts per target for each of a set of targets of interest, 120,000 counts per target for each of a set of targets of interest, 130,000 counts per target for each of a set of targets of interest, 140,000 counts per target for each of a set of targets of interest, 150,000 counts per target for each of a set of targets of interest, 160,000 counts per target for each of a set of targets of interest, 170,000 counts per target for each of a set of targets of interest, 180,000 counts per target for each of a set of targets of interest, 190,000 counts per target for each of a set of targets of interest, 200,000 counts per target for each of a set of targets of interest, 210,000 counts per target for each of a set of targets of interest, 220,000 counts per target for each of a set of targets of interest, 230,000 counts per target for each of a set of targets of interest, 240,000 counts per target for each of a set of targets of interest, 250,000 counts per target for each of a set of targets of interest, 260,000 counts per target for each of a set of targets of interest, 270,000 counts per target for each of a set of targets of interest, 280,000 counts per target for each of a set of targets of interest, 290,000 counts per target for each of a set of targets of interest, 300,000 counts per target for each of a set of targets of interest, or other counts per target for each of a set of targets of interest.

In relation to accurate, fast, and precise counting across the entire dynamic range, if the number of targets of the sample is within a first range, generating the count can include performing a first set of steps, if the number of targets of the sample is within a second range, generating the count comprises performing a second set of steps different than the first set of steps, and if the number of targets of the sample is within a third range, generating the count comprises generating a third set of steps different than the first set of steps and the second set of steps. The first range can be a range from a lower order of magnitude of 1 molecule to a higher order of magnitude of 10² molecules. The second range can be a range from a lower order of magnitude of 10² molecules to a higher order of magnitude of 10⁵ molecules. The third range can be a range from a lower order of magnitude of 10⁵ molecules to a higher order of magnitude of 10⁶ molecules or greater. However, variations of the methods can involve utilizing different counting approaches within other ranges of target numbers.

Relatedly, an aspect of the disclosure provides embodiments, variations, and examples of devices and methods for rapidly generating partitions (e.g., droplets from a sample fluid, droplets of an emulsion) and distributing sample material across partitions, where, the device includes: a first substrate defining a reservoir comprising a reservoir inlet and a reservoir outlet; a membrane coupled to the reservoir outlet and comprising a distribution of holes; and a supporting body comprising an opening configured to retain a collecting container in alignment with the reservoir outlet. During operation, the first substrate can be coupled with the supporting body and enclose the collecting container, with the reservoir outlet aligned with and/or seated within the collecting container. During operation, the reservoir can contain a sample fluid (e.g., a mixture of nucleic acids of the sample and materials for an amplification reaction), where application of a force to the device or sample fluid generates a plurality of droplets within the collecting container at an extremely high rate (e.g., of at least 200,000 droplets/minute, of at least 300,000 droplets/minute, of at least 400, droplets/minute, of at least 500,000 droplets/minute, of at least 600,000 droplets/minute, of at least 700,000 droplets/minute, of at least 800,000 droplets/minute, of at least 900,000 droplets/minute, of at least 1 million droplets/minute, of at least 2 million droplets/minute, of at least 3 million droplets/minute, of at least 4 million droplets/minute, of at least 5 million droplets/minute, of at least 6 million droplets per minute, etc.), where the droplets are stabilized in position (e.g., in a close-packed format, in equilibrium stationary positions) within the collecting container.

In embodiments, the sample and/or targets of the sample can include or otherwise be derived from whole tissue structures, tissue portions (e.g., histological tissue slices, formalin-fixed paraffin-embedded (FFPE) tissue, frozen tissue, biopsied tissues, fresh frozen plasma, seeded natural scaffolds, seeded synthetic scaffolds, etc.), organs, whole organisms, organoids, cell suspensions (e.g., frozen cell suspensions that are separated prior to processing with the system, a cell suspension, a nuclei suspension, single cells, organelles, sub-organelle structures, intra-organelle components, viruses, microorganisms, and other samples.

An aspect of the disclosure provides embodiments, variations, and examples of a method for rapidly generating partitions (e.g., droplets from a sample fluid, droplets of an emulsion) within a collecting container at an extremely high rate, each of the plurality of droplets including an aqueous mixture for a digital analysis, wherein upon generation, the plurality of droplets is stabilized in position (e.g., in a close-packed format, at equilibrium stationary positions, etc.) within a continuous phase (e.g., as an emulsion having a bulk morphology defined by the collecting container). In aspects, partition generation can be executed by driving the sample fluid through a distribution of holes of a membrane, where the applied force can be one or more of centrifugal (e.g., under centrifugal force), associated with applied pressure, magnetic, or otherwise physically applied.

In relation to a single-tube workflow in which the collecting container remains closed (e.g., the collecting container has no outlet, there is no flow out of the collecting container, to avoid sample contamination), method(s) can further include transmitting heat to and from the plurality of droplets within the closed collecting container according to an assay protocol. In relation to generation of emulsions having suitable clarity (e.g., without refractive index matching), method(s) can further include transmission of signals from individual droplets from within the closed collecting container, for readout (e.g., by an optical detection platform, by another suitable detection platform).

Where method(s) include transmitting heat to and from the plurality of droplets, within the closed container, the droplets are stable across a wide range of temperatures (e.g., 1° C. through 95° C., greater than 95° C., less than 1° C.) relevant to various digital analyses and other bioassays, where the droplets remain consistent in morphology and remain unmerged with adjacent droplets.

The disclosure generally provides mechanisms for efficient capture, distribution, and labeling of target material (e.g., DNA, RNA, miRNA, proteins, small molecules, single analytes, multianalytes, etc.) in order to enable genomic, proteomic, and/or other multi-omic characterization of materials, in parallel and in a multiplexed manner, for various applications.

Another aspect of the present disclosure provides a non-transitory computer readable medium comprising machine executable code that, upon execution by one or more computer processors, implements any of the methods above or elsewhere herein.

The disclosure provides compositions, methods, and systems for multiplexed detection of targets that can provide value in research or other non-clinical settings, with or without evaluation and processing of live human or mammalian biological material, and without the immediate purpose of obtaining a diagnostic result of a disease or health condition.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties for all purposes and to the same extent as if each individual publication, patent, or patent application was specifically and individually indicated to be incorporated by reference.

Furthermore, where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

### BRIEF DESCRIPTION OF THE DRAWINGS

FIG. 1A depicts a flowchart of an embodiment of a method for high dynamic range digital quantitation of targets.

FIG. 1B depicts a flowchart of portions of an embodiment of a method for high dynamic range digital quantitation of targets.

FIG. 2 depicts a schematic of components implemented in an embodiment of a method for high dynamic range digital quantitation of targets.

FIG. 3 depicts a schematic of an embodiment of a system for partitioning samples.

FIG. 4 depicts schematics of representative directions along which characterization of partitions as positive partitions and/or negative partitions can be analysed.

FIGS. 5A-5D depict control and/or training data images of representative positive partitions and negative partitions, where the images depict two-dimensional cross sections from single droplets.

FIG. 6A depicts an example of droplet feature characterization models used to identify signal-positive and/or signal-negative droplets of a sample.

FIG. 6B depicts an example of classification architecture and training used to generate counts of sample targets.

FIG. 7A depicts a flowchart of an example of a method for high dynamic range digital quantitation of targets, across different ranges of the dynamic range.

FIG. 7B depicts a flowchart of a variation of a method for high dynamic range digital quantitation of targets, with improved performance.

FIG. 7C depicts exemplary method variations for further improving performance of high dynamic range digital quantitation of targets, across a wide dynamic range.

FIG. 7D depicts an example of subsampled regions of a cross-section of readout image data, in relation to improving performance of high dynamic range digital quantitation of targets.

FIG. 8 illustrates a computer system that is programmed or otherwise configured to implement methods provided herein.

### DETAILED DESCRIPTION OF THE INVENTION(S)

While various embodiments of the invention have been shown and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

### 1. General Overview

The invention(s) described can confer several benefits over conventional systems, methods, and compositions.

The present disclosure covers systems, devices, methods performed by such systems and devices, and methods of manufacturing and assembling such devices. Generally, embodiments of the device include assemblies of reservoirs, functionalized membranes, and supporting bodies for collecting containers, where the assemblies rapidly produce droplets of an emulsion for digital analyses or other applications of use. Droplets produced by such devices are stabilized in a three dimensional format within closed collecting containers, thereby providing a “single-tube” workflow that eliminates risk of sample cross-contamination from initial reception of a sample, to distributing of the sample across an extremely high number of partitions, to performance of reactions within individual partitions, to detecting signals generated by contents of individual partitions from with the closed collecting containers.

The systems, methods, and devices disclosed herein can provide several additional benefits over other systems and methods, and such systems, methods, and devices are further implemented into many practical applications across various disciplines.

Devices, methods, and systems of the present disclosure may generate a plurality of droplets at an extremely high rate, where the droplets are stabilized in position (e.g., in a close-packed format, in equilibrium stationary positions) within a collecting container. Notably, the droplets are stable across a wide range of temperatures (e.g., 1° C. through 95° C., greater than 95° C., less than 1° C.) relevant to various digital analyses and other bioassays, where the droplets remain consistent in morphology and remain unmerged with adjacent droplets. Stabilization of the droplets within a continuous phase of an emulsion is further performed in a manner where the emulsion has a high degree of clarity (e.g., greater than 50% transmissivity of light, greater than 60% transmissivity of light, greater than 70% transmissivity of light, greater than 80% transmissivity of light, greater than 90% transmissivity of light, greater than 99% transmissivity of light, etc.), such that signals from cross-sections of the emulsion within the collecting container can be interrogated (e.g., using a 3D imaging technique, using a planar imaging technique, etc.).

The devices, systems, and methods disclosed herein can further generate droplets in a consistent and controlled manner (e.g., as monodisperse, uniform droplets with a low degree of polydispersity) for applications in biotechnology (e.g., with respect to microscale and nanoscale assays) of other fields.

In particular, the invention(s) enable detection and digital quantitation of a set of targets of a sample, with the ability to count targets of the sample across a wide dynamic range (e.g., of 6 logarithms or greater), with unprecedented accuracy and precision across the entire dynamic range.

In the context of digital multiplexed analyses, the disclosure provides systems, methods, and compositions that can achieve counting performance with a wide dynamic range, due to the number of partitions involved and occupancy of the partitions by targets of the sample. The invention(s) enable quantitation of targets across a dynamic range of five logarithms, across a dynamic range of six logarithms, across a dynamic range of seven logarithms, across a dynamic range of eight logarithms, or greater. In embodiments, the invention(s) described can enable precise and accurate counting of more than one target of a sample, more than 100 targets of a sample, more than 1,000 targets of a sample, more than 10,000 targets of a sample, more than 100,000 targets of a sample, more than 1,000,000 targets of a sample, more than 10,000,000 targets of a sample, or more than 100,000,000 targets of a sample, where the targets are individually distributed across partitions, such that no partition contains more than one target (e.g., each partition has zero or one target).

The invention(s) enable generation of counts of targets of a sample, across a wide dynamic range, with an accuracy greater than 90%, an accuracy greater than 91%, an accuracy greater than 92%, an accuracy greater than 93%, an accuracy greater than 94%, an accuracy greater than 95%, an accuracy greater than 96%, an accuracy greater than 97%, an accuracy greater than 98%, an accuracy greater than 99%, an accuracy greater than 99.5%, an accuracy greater than 99.9%, or greater. Accuracy can be determined based upon comparing determined counts and expected or known counts for a sample (e.g., for a test sample where the number of targets is known prior to processing the sample according to aspects of the inventions described).

The invention(s) enable generation of counts of targets of a sample, across a wide dynamic range, with a precision characterized by a coefficient of variation less than 10%, characterized by a coefficient of variation less than 9%, characterized by a coefficient of variation less than 8%, characterized by a coefficient of variation less than 7%, characterized by a coefficient of variation less than 6%, characterized by a coefficient of variation less than 5%, characterized by a coefficient of variation less than 4%, characterized by a coefficient of variation less than 3%, characterized by a coefficient of variation less than 2%, characterized by a coefficient of variation less than 1%, or better. In variations, precision can be characterized in relation to another measure of statistical variability.

The invention(s) can also involve readout of signal-positive partitions upon scanning a set of cross sections of a closed collecting container containing the set of partitions, for each of a set of color channels.

The invention(s) also involve high performance generation of counts from readout data, where generation of counts can be performed within 20 seconds, within 15 seconds, within 10 seconds, within 9 seconds, within 8 seconds, within 7 seconds, within 6 seconds, within 5 seconds, within 4 seconds, within 3 seconds, within 2 seconds, within 1 second, within 0.5 seconds, within 0.1 seconds, or less.

The invention(s) also involve generation of an emulsion having a set of droplets (e.g., as a dispersed phase), within a continuous phase, wherein each of the set of droplets contains zero or one target of the sample (i.e., no droplet of the set of droplets contains two or more targets of the sample). In embodiments, droplets of the set of droplets are aqueous (e.g., derived from the sample), and are each surrounded by a thin film of liquid that is immiscible with the aqueous droplets. The continuous phase of the emulsion is also aqueous.

The invention(s) also involve generation of a clear emulsion that facilitates optical interrogation without requiring transmission of droplets of the sample individually through microfluidic structures. In one variation, clarity can be characterized in relation to transmissivity as detectable by a transmission detector and/or in relation to a suitable distance or depth (e.g., depth or distance into a collecting container for the emulsion; through a depth of a container of the emulsion, along an axis in which measurement of clarity is performed, etc.), where, in variations, the threshold level of clarity of the emulsion is associated with a transmissivity greater than 70% transmissivity, greater than 80% transmissivity, greater than 90% transmissivity, greater than 95% transmissivity, greater than 99% transmissivity, etc. As such, in accordance with methods described, upon measuring clarity of the emulsion (e.g., later stage emulsion) using a transmission detector the emulsion is characterized by a clarity associated with greater than 70% transmissivity, greater than 80% transmissivity, greater than 90% transmissivity, greater than 95% transmissivity, greater than 99% transmissivity, etc., which is above the threshold level of clarity.

The invention(s) also involve generation of a gel-like emulsion. In embodiments, the emulsion having gel-like properties cannot or does not flow (e.g., through microfluidic structures) upon generation. In embodiments, generation of the emulsion produces droplets of the emulsion within a closed collecting container, where the closed collecting container remains closed throughout optical interrogation of the emulsion for detection of targets. In embodiments, the emulsion is stabilized in position within the closed collecting container upon generation of the emulsion, such that droplets of the emulsion do not move relative to each other and/or relative to the closed collecting container upon generation and stabilization in position within the closed collecting container. Such gel-like properties enables repeat scanning of each droplet using different interrogation channels, where a compiled optical signature can be aggregated from optical signatures transmitted from each droplet, where individual optical signatures are generated using each of a set of interrogation channels. Each of the set of interrogation channels can have its own excitation profile and emission profile.

Gel-like aspects of the emulsion can be characterized in terms of shear-thickening/dilatancy behavior, viscosity, modulus, or other mechanical parameters. In embodiments, a gel-like emulsion generated according to methods described can have a viscosity of greater than 100 centipoise, greater than 1000 centipoise, greater than 10,000 centipoise, greater than 50,000 centipoise, greater than 100,000 centipoise, or greater.

Performance values described are achieved in part due to the high number of partitions involved when using the systems, methods, and compositions described, where distribution of sample targets across partitions results in low occupancy of partitions by targets, and large partition numbers contribute to significantly low or non-existent percentages of doublets (e.g., single partitions occupied by two targets), triplets (e.g., single partitions occupied by three targets), or other forms of multi-plets (single partitions occupied by multiple targets). In particular, successful characterization across such a wide dynamic range is attributed to the high degree of partitioning (with achievable numbers of generated partitions described) and extremely low occupancy (with achievable percent occupancies described), such that multiple molecules from the target molecules of interest have a minimal (or zero) probability of occupying the same partition as another target molecule. In such a high-partition and low-occupancy regime, there is no competition associated with multiple target molecules per partition, and the platform is not subject to problems related to differences in PCR efficiency between different target molecules.

In relation to accurate, fast, and precise counting across the entire dynamic range, the invention(s) involve applying appropriate counting methods to numbers of targets within different ranges of the entire dynamic range available. For instance, if the number of targets of the sample is within a first range, generating the count can include performing a first set of steps, if the number of targets of the sample is within a second range, generating the count comprises performing a second set of steps different than the first set of steps, and if the number of targets of the sample is within a third range, generating the count comprises generating a third set of steps different than the first set of steps and the second set of steps. The first range can be a range from a lower order of magnitude of 1 molecule to a higher order of magnitude of 10² molecules. The second range can be a range from a lower order of magnitude of 10² molecules to a higher order of magnitude of 10⁵ molecules. The third range can be a range from a lower order of magnitude of 10⁵ molecules to a higher order of magnitude of 10⁶ molecules or greater. However, variations of the methods can involve utilizing different counting approaches within other ranges of target numbers.

Additionally or alternatively, the invention(s) can provide functionality for detection of target analytes in a differentiable and multiplexed manner. In examples, analytes can include one or more of: DNA, RNA, miRNA, proteins, small molecules, single analytes, multianalytes, chemicals, and/or other analytes. For nucleic acid targets, capture probes of compositions described can include complementary molecules to the nucleic acid targets. For protein targets or small molecule targets, capture probes of the compositions described can include antibodies or aptamers conjugated with specific nucleic acid sequences for detection.

The invention(s) can be applied to samples from human organisms, other multicellular animals, plants, fungi, unicellular organisms, viruses, and/or other material, with respect to evaluating presence or absence of sets of targets in parallel. Characterizations of the sets of targets can then be used for diagnostic purposes and/or for generation of targeted therapies to improve states of organisms from which the samples were sourced. The invention(s) can also provide value in research or other non-clinical settings, with or without evaluation and processing of live human or mammalian biological material, and without the immediate purpose of obtaining a diagnostic result of a disease or health condition.

The invention(s) confer(s) the benefit of providing non-naturally occurring compositions for facilitating interactions with and amplification of a large set of target analytes from a sample in parallel, with improved efficiency, without utilizing complex microfluidic setups, and in a manner that reduces overall costs. As such, the invention(s) provide a cost-competitive alternative to other methods for detection and digital quantitation of a large number of target analytes in a multiplexed manner.

The invention(s) can provide mechanisms for target-specific/allele-specific amplification and can be applied to digital polymerase chain reaction (dPCR) and/or other PCR-associated assays.

Additionally or alternatively, the invention(s) can confer any other suitable benefit.

### 2. Methods and Materials

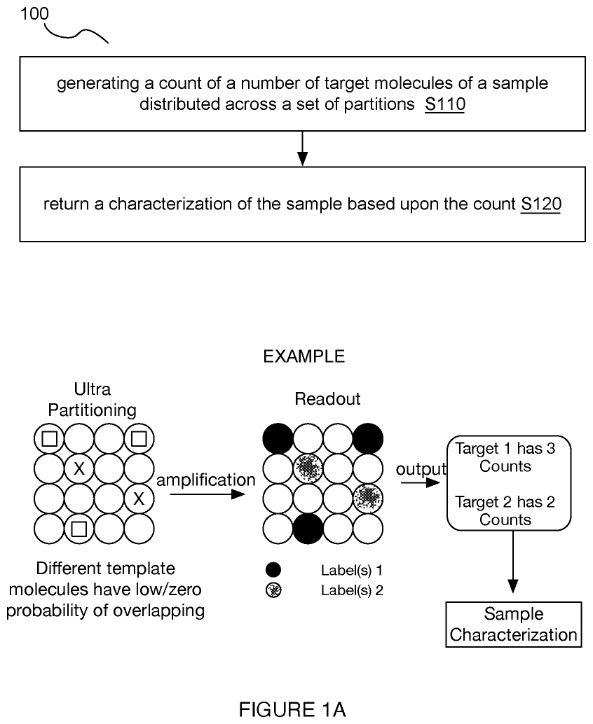

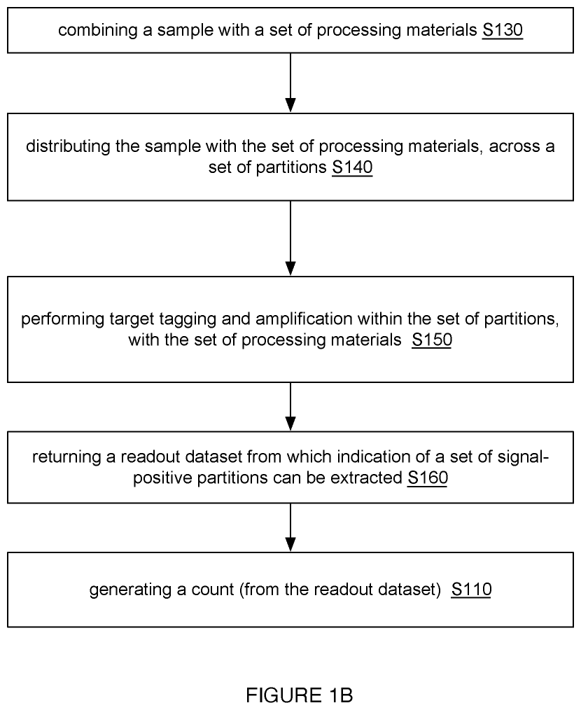

As shown in FIG. 1A, embodiments of a method 100 include: generating a count of a number of target molecules of a sample distributed across a set of partitions (e.g., a high number of partitions at low occupancy) S110; and returning a characterization of the sample based upon the count S120. The method 100 functions to achieve high dynamic range counting of a number of targets potentially represented in a sample, and in variations, can provide unprecedented performance in relation to dynamic counting range, precision, accuracy, and speed of generating the count.

In variations, the method 100 provides quantitation of targets across a dynamic range of five logarithms, across a dynamic range of six logarithms, across a dynamic range of seven logarithms, across a dynamic range of eight logarithms, or greater. As such, the methods described enable precise and accurate counting of more than one target of a sample, more than 100 targets of a sample, more than 1,000 targets of a sample, more than 10,000 targets of a sample, more than 100,000 targets of a sample, more than 1,000,000 targets of a sample, more than 10,000,000 targets of a sample, or more than 100,000,000 targets of a sample, where the targets are individually distributed across partitions, such that no partition contains more than one target (e.g., each partition has zero or one target, each partition has less than two targets).

Embodiments of the method 100 described enable generation of counts of targets of a sample, across a wide dynamic range, with an accuracy greater than 90%, an accuracy greater than 91%, an accuracy greater than 92%, an accuracy greater than 93%, an accuracy greater than 94%, an accuracy greater than 95%, an accuracy greater than 96%, an accuracy greater than 97%, an accuracy greater than 98%, an accuracy greater than 99%, an accuracy greater than 99.5%, an accuracy greater than 99.9%, or greater. Accuracy can be determined based upon comparing determined counts and expected or known counts for a sample (e.g., for a test sample where the number of targets is known prior to processing the sample according to aspects of the inventions described).

Embodiments of the method 100 described enable generation of counts of targets of a sample, across a wide dynamic range, with a precision characterized by a coefficient of variation less than 10%, characterized by a coefficient of variation less than 9%, characterized by a coefficient of variation less than 8%, characterized by a coefficient of variation less than 7%, characterized by a coefficient of variation less than 6%, characterized by a coefficient of variation less than 5%, characterized by a coefficient of variation less than 4%, characterized by a coefficient of variation less than 3%, characterized by a coefficient of variation less than 2%, characterized by a coefficient of variation less than 1%, or better. In variations, precision can be characterized in relation to another measure of statistical variability.

Embodiments of the method 100 described enable generation of counts from readout data (e.g., readout data from which indications of signal-positive partitions can be extracted), where generation of counts can be performed within 20 seconds, within 15 seconds, within 10 seconds, within 9 seconds, within 8 seconds, within 7 seconds, within 6 seconds, within 5 seconds, within 4 seconds, within 3 seconds, within 2 seconds, within 1 second, within 0.5 seconds, within 0.1 seconds, or less.

One or more of the methods described can further include steps for processing a sample or providing an environment for producing the processed sample such that it can produce signals for generation of readout data, where counts of targets can be generated from the readout data.

Processing the sample can include (as shown in FIG. 1B): combining a sample with a set of processing materials for a set of reactions (e.g., a target-tagging reaction, an amplification reaction, etc.) S130; distributing the sample with the set of processing materials, across a set of partitions (e.g., a high number of partitions at low occupancy, such that different targets of the set of targets do not co-inhabit a single partition) S140; performing target tagging and amplification, with the set of processing materials S150; returning a readout dataset from which indication of a set of signal-positive partitions can be extracted S160; and generating a count of the number of targets from the readout dataset S110.

The method(s) described can be implemented using embodiments, variations, and examples of system components described in U.S. application Ser. No. 17/230,907 filed on 14 Apr. 2021 and/or U.S. application Ser. No. 17/687,080 filed 4 Mar. 2022, which are each hereby incorporated in its entirety by this reference. Additionally or alternatively, the method(s) can be implemented using other system elements.

Sample Types and Targets: In variations, the method 100 can be used to process sample types including biological fluids including or derived from one or more of: blood (e.g., whole blood, peripheral blood, non-peripheral blood, blood lysate, etc.), plasma, serum, saliva, reproductive fluids, mucus, pleural fluid, pericardial fluid, peritoneal fluid, amniotic fluids, otic fluid, sweat, interstitial fluid, synovial fluid, cerebral-spinal fluid, urine, gastric fluids, biological waste, other biological fluids; tissues (e.g., homogenized tissue samples); single cells; nuclei; food samples; liquid consumable samples; and/or other sample materials. Samples can be derived from human organisms, other multicellular animals, plants, fungi, unicellular organisms, viruses, and/or other material. In specific examples, samples processed can include maternal samples (e.g., blood, plasma, serum, urine, chorionic villus, etc.) including maternal and fetal material (e.g., cellular material, cell-free nucleic acid material, other nucleic acid material, etc.) from which prenatal detection or diagnosis of genetic disorders (e.g., aneuploidies, genetically inherited diseases, other chromosomal issues, etc.) can be performed.

In embodiments, targets detected and/or quantified according to embodiments, variations, and examples of the method 100 can include: nucleic acids (e.g., DNA, RNA, miRNA, etc.), proteins, amino acids, peptides, small molecules, single analytes, multianalytes, chemicals, and/or other target material, in order to enable genomic, proteomic, and/or other multi-omic characterizations and diagnoses for various applications. Genetic targets can include one or more of: single nucleotide polymorphisms (SNPs), copy number variations (CNVs), insertions, deletions, genes, methylated loci, and/or other loci of interest.

Additionally or alternatively, in other specific applications, target material tagged and evaluated according to methods described can provide diagnostics and/or characterizations in relation to one or more of: monitoring or detection of products (e.g., proteins, chemicals) released from single cells (e.g., interleukins or other compounds released from immune cells); monitoring cell survival and/or division for single cells; monitoring or detection of enzymatic reactions involving single cells; antibiotic resistance screening for bacteria; characterization of pathogens in a sample (e.g., in relation to infections, sepsis, in relation to environmental and food samples, etc.); microbiome characterizations (e.g., based upon detection of hypervariable regions of rRNA); characterization of heterogeneous cell populations in a sample; characterization of individual cells or viral particles; characterization of individual nuclei; monitoring of viral infections of a single host cell; liquid biopsies and companion diagnostics; detection of cancer forms from various biological samples (e.g., from cell-free nucleic acids, tissue biopsies, biological fluids, feces, etc.) based upon characterization of target panels; detection and/or monitoring of minimal residual diseases; monitoring responses to therapies; detection or prediction of rejection events of transplanted organs; other diagnostics associated with other health conditions; other characterizations of statuses of other organisms; and other suitable applications.

Counting targets according to methods described can include generating counts of different targets that are individually and spatially segregated within different partitions, and differentially tagged (e.g., with label combinatorics), using embodiments, variations, and examples of method steps and system components described in U.S. application Ser. No. 18/085,217 filed on 20 Dec. 2022 which is hereby incorporated in its entirety by this reference. As such, embodiments, variations, and examples of the method 100 described herein can include generating counts of different subsets of targets potentially represented in a sample, based upon differential tagging of targets of the different subsets of targets.

### 2.1 Method—Assay Materials and Compositions

### 2.1.1 Method—Assay Materials and Compositions for Competitive Target-Specific Assays

Step S130 recites: combining the sample with a set of processing materials, which functions to tag and amplify targets of the sample, where tagging enables subsequent detection of targets captured within individual partitions, using interrogation techniques described in more detail below.

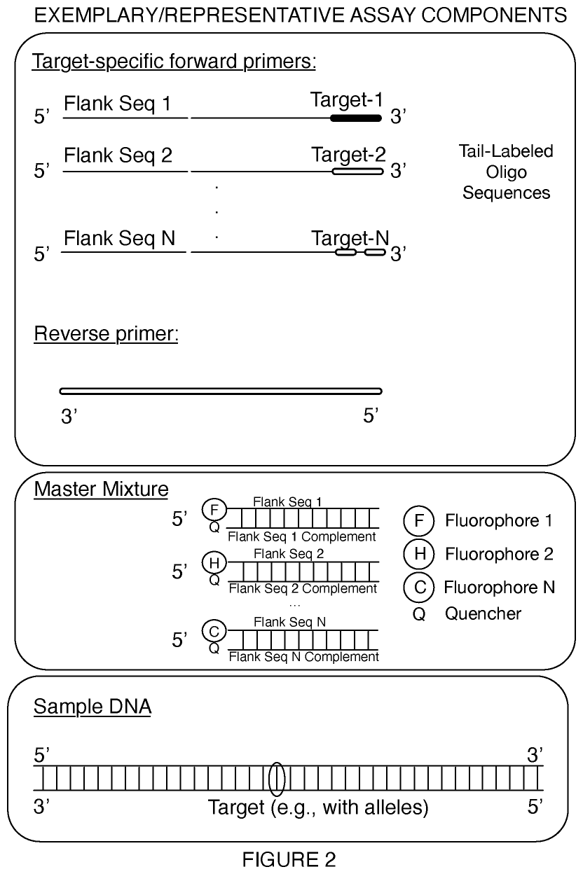

In embodiments, the set of processing materials can include: a) for each of the set of targets potentially represented in the sample, primers (e.g., forward and reverse primers) for amplifying the target(s) of the sample, and b) a master mixture for amplification and tagging reactions (e.g., using label combinatorics). In variations, the set of processing materials can include: a) for each of the set of targets potentially represented in the sample, a set of target-specific forward primers corresponding to a respective target of the set of targets, and a common reverse primer for the set of target-specific forward primers, and b) a master mixture including amplification reagent as well as: for each of the set of targets, a set of target-specific flanking sequences corresponding to different targets of the set of targets, as shown in FIG. 2.

Concentrations of forward primers can range from 50 nM to 300 nM in solution, or alternatively, less than 50 nM or greater than 300 nM in solution. Concentrations of reverse primers can range from 100 nM to 600 nM in solution, or alternatively, less than 100 nM or greater than 600 nM in solution. Concentrations of reporter oligonucleotides (e.g., fluorescent reporter oligonucleotides) can range from 30 nM to 200 nM in solution, or alternatively, less than 30 nM or greater than 200 nM in solution. Concentrations of quencher oligonucleotides can range from 100 nM to 600 nM in solution, or alternatively, less than 100 nM or greater than 600 nM in solution.

Primers (e.g., forward primers, reverse primers) can have lengths of 20 base pairs, 21 base pairs, 22 base pairs, 23 base pairs, 24 base pairs, 25 base pairs, 26 base pairs, 27 base pairs, 28 base pairs, 29 base pairs, 30 base pairs, 35 base pairs, 40 base pairs, 45 base pairs, 50 base pairs, an intermediate number of base pairs, or a greater number of base pairs. In variations, primers can incorporate sequence regions corresponding to probes and target sequences (e.g., a 20 base pair target sequence, a target sequence having another suitable length, etc.), and be designed for various levels of plexy (e.g., 1-plex conditions, 2-plex conditions, 3-plex conditions, 4-plex conditions, 5-plex conditions, 6-plex conditions, 7-plex conditions, etc.) as described. In variations, forward primers can be longer than reverse primers, and in specific examples, used of forward primers having lengths 5-10 base pairs longer (e.g., than reverse primers, than another reference length) produced higher counts (e.g., 8-10% higher counts) and higher SNR values (e.g., 12-17% higher SNR values) in relation to shorter primer lengths, when detecting of targets from partitions, thereby providing higher detection performance.

Primers (e.g., forward primers, reverse primers) can have annealing temperatures from 48 C-65 C or another suitable annealing temperature range based upon reactions performed according to various assays. Primers (e.g., forward primers, reverse primers) can have melting temperatures from 65 C to 70 C (e.g., from 67 C to 68.8 C) or another suitable melting temperature range based upon reactions performed according to various assays.

Characteristics of forward and reverse primers described above can be reversed (e.g., the set of processing materials can include a forward primer and a set of target-specific reverse primers). Still alternatively, both forward and reverse primers can be target-specific.

As noted briefly above and shown in FIG. 2, in embodiments, the master mixture can include amplification reagents and, for each of the set of targets, a set of target-specific flanking sequences corresponding to different targets of the set of targets, in order to support multiplexed processing, detection, and digital quantitation. As such, in one variation, the set of processing materials can include, for a target of the set of targets: a primer set comprising: a common primer and a set of target-specific primers structured to interact with a target region of the target, the set of target-specific primers comprising a first target-specific primer comprising a first flanking sequence, and a first fluorophore-labeled oligonucleotide corresponding to the flanking sequence, the first fluorophore-labeled oligonucleotide comprising a first fluorophore configured to transmit a first target signal if the target region is amplified.

For tagging a target with probes configured to emit multiple colors (where tandem probes are described in more detail below), the set of target-specific primers can further include a second target-specific primer comprising a second flanking sequence, and the set of processing materials further comprises a second fluorophore-labeled oligonucleotide corresponding to the second flanking sequence, the second fluorophore-labeled oligonucleotide comprising a second fluorophore configured to transmit a second target signal if the target region is amplified, such that the target can be positively detected based upon the first target signal and the second target signal. Alternatively, a single primer can be used to tag the target, along with tandem adapters corresponding to the probes used to tag the targets. As such, the set of processing materials can include at least one primer structured to tag the target with a first probe having a first fluorophore and a second probe having a second fluorophore (and/or additional probes with additional fluorophores), wherein the first fluorophore and the second fluorophore (and optional additional fluorophores) correspond to two (or more) color channels of the number of color channels.

The master mixture can include a probe including a dye/fluorophore with complementary quencher for each target, a polymerase (e.g., Taq polymerase), dNPTs, and buffer components.

With respect to tagging implemented using the forward primers, and corresponding dyes/fluorophore families of probes, dyes/fluorophores can be associated with chemical families including: acridine derivatives, arylmethine derivatives, fluorescein derivatives, anthracene derivatives, tetrapyrrole derivatives, xanthene derivatives, oxazine derivatives, dipyrromethene derivatives, cyanine derivatives, squaraine derivates, squaraine rotaxane derivatives, naphthalene derivatives, coumarin derivatives, oxadiazole derivatives, pyrene derivatives, and/or other chemicals. Such fluorophores can further be attached to other functional groups as needed for tagging of targets in a detectable manner.

In examples, dyes (e.g., for tagging of RNAs, DNAs, oligonucleotides, etc.) can include one or more of: FAM, (e.g., 6-FAM), Cy3TM, Cy5TM, Cy5.5TM, TAMRATM (e.g., 5-TAMRA, 6-TAMRA, etc.), MAX, JOE, TETTM, ROX, TYETM (e.g., TYE 563, TYE 665, TYE 705, etc.), Yakima Yellow®, HEX, TEX (e.g., TEX 615), SUN, ATTOTM (e.g., ATTO 488, ATTO 532, ATTO 550, ATTO 565, ATTO Rho101, ATTO 590, ATTO 633, ATTO 647, etc.), Alexa Fluor® (e.g., Alexa Fluor 488, Alexa Fluor 532, Alexa Fluor 546, Alexa Fluor 594, Alexa Fluor 647, Alexa Fluor 660, Alexa Fluor 750, etc.), IRDyes® (e.g., 5′IRDye 700, 5′IRDye 800, 5′IRDye 800 CW, etc.), Rhodamine (e.g., Rhodamine Green, Rhodamine Red, Texas Red®, Lightcycler®, Dy 750, Hoechst dyes, DAPI dyes, SYTOX dyes, chromomycin dyes, mithramycin dyes, YOYO dyes, ethidium bromide dyes, acridine orange dyes, TOTO dytes, thiazole dyzes, CyTRAK dyes, propidium iodide dyes, LDS dyes, BODIPY dyes, and/or other dyes.

In examples, cell function dyes for tagging of target material and detection can include one or more of: DCFH, DHR, SNARF, indo-1, Fluo-3, Fluo-4, and/or other dyes.

In examples, fluorescent proteins for tagging of target material and detection can include one or more of: cerulean, mCFP, mTurquoise, T-Sapphire, CyPet, ECFP, CFP, EBFP, Azurite, and/or other fluorescent proteins.

Dyes/fluorophores implemented can correspond to wavelength ranges in the visible spectrum and/or non-visible spectrum of electromagnetic radiation. Furthermore, dyes/fluorophores implemented can be configured to prevent overlapping wavelengths (e.g., of emission) and/or signal bleed through with respect to multiplexed detection and achieving high SNR values involving detection of signals from packed partitions. In variations, the set of processing materials can include components for 7 wavelength ranges for multiplexed detection of targets; however, the set of processing materials can include components for less than 7 wavelength ranges (e.g., one wavelength, two wavelengths, three wavelengths, four wavelengths, five wavelengths) or more than 7 wavelength ranges.

Quencher oligonucleotides implemented can include a quencher molecule configured such that, when the quencher oligonucleotide anneals with a primer having a fluorophore, the quencher molecule is in proximity to (e.g., directly opposite) the fluorophore in order to quench the fluorophore. Additionally or alternatively, quenchers can include one or more of: black hole quenchers, static quenchers, self-quenchers (e.g., fluorophores that self-quench under certain conditions by producing secondary structures or other structures), and/or other suitable quenchers. Variations of positions of quenchers (e.g., when tandem probes are involved) are described in more detail below.

The set of processing materials of Step S130 can additionally or alternatively include implementation of components structured to improve signal-to-noise ratio (SNR) characteristics in the context of multiplexed detection, by increasing signal characteristics and/or reducing background (e.g., noise other artifacts). The components can include one additive for each wavelength range/color for detection (as opposed to one additive for each target/SNP being evaluated). Additionally or alternatively, the additives can have from 5-20 bases or another suitable number of bases. Additionally or alternatively, modified nucleic acids (e.g., such as locked nucleic acids (LNA) or other modified nucleic acids) can be incorporated into forward and/or reverse primers of the set of processing materials to improve SNR. In variations, LNA content can occupy a percentage (e.g., 10-60% LNA content) of the respective primer to improve SNR, where LNA content can be biased toward the 3′ end, the 5′ end, or intermediate the 3′ and 5′ ends.

However, the set of processing materials can additionally or alternatively include other suitable components and/or be configured in another suitable manner.

Furthermore, with respect to different wavelength ranges, different targets can be tagged with dye/fluorophore colors in a manner that promotes discrimination of results (e.g., without overlap) upon detection of signals from processed sample material. Furthermore, different targets can be matched with different combinations of colors/associated wavelengths in order to provide distinction upon detection of signals from processed sample materials. Variations and examples of multiplexing based upon color combinatorics and other features are further provided in Applications incorporated by reference.

### 2.2 Method—Partitioning of Sample with Processing Materials

Distributing the sample combined with the set of processing materials, across a set of partitions in step S140 can include receiving a sample (variations and examples of which are described above) at a vessel passively or actively (e.g., with applied force, such as with gravitational force, with centrifugal force, with pressurization, etc.). The sample and processing materials can be delivered manually (e.g., with a fluid aspiration and delivery device, such as a pipettor). The sample and processing materials can additionally or alternatively be delivered with automation (e.g., using liquid handling apparatus or other sample handling apparatus).

In variations, vessel formats can include: tubes (e.g., PCR tubes) containing partitions of the sample (e.g., in droplet format, in emulsion format, in another format), wells (e.g., microwells, nanowells, etc.), channels, chambers, and/or other suitable containers. Additionally or alternatively, alternative variations of step S140 can include receiving the sample at other suitable substrates (e.g., slides, plates, etc.) functionalized with material components structured to interact with target material of the sample. For instance, sample material can be spotted onto substrates with material components structured to interact with target material of the sample and in a detectable manner.

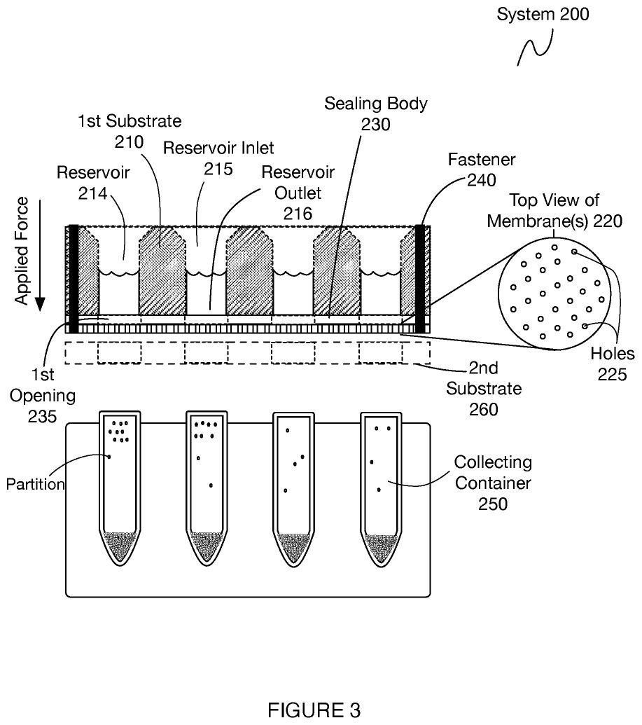

Embodiments, variations, and examples of the methods described can be implemented by or by way of embodiments, variations, and examples of components of system 200 shown in FIG. 3, with a first substrate 210 defining a set of reservoirs 214 (for carrying sample/mixtures for droplet generation), each having a reservoir inlet 215 and a reservoir outlet 216; one or more membranes (or alternatively, droplet-generating substrates) 220 positioned adjacent to reservoir outlets of the set of reservoirs 214, each of the one or more membranes 220 including a distribution of holes 225; and optionally, a sealing body 230 positioned adjacent to the one or more membranes 120 and including a set of openings 235 aligned with the set of reservoirs 214; and optionally, one or more fasteners (including fastener 240) configured to retain the first substrate 210, the one or more membranes 220, and optional sealing body 230 in position relative to a set of collecting containers 250. In variations, the system 200 can additionally include a second substrate 260, wherein the one or more membranes 220 and optionally, the sealing body 230, are retained in position between the first substrate 210 and the second substrate 260 by the one or more fasteners. In using embodiments, variations, and examples of the system 200, material derived from each sample is retained in its own tube and does not require batching and pooling, allowing for scalable batch size.

In variations, the distribution of holes 225 can be generated in bulk material with specified hole diameter(s), hole depth(s) (e.g., in relation to membrane thickness), aspect ratio(s), hole density, and hole orientation, where, in combination with fluid parameters, the structure of the membrane can achieve desired flow rate characteristics, with reduced or eliminated polydispersity and merging, suitable stresses (e.g., shear stresses) that do not compromise the single cells but allow for partitioning of the single cells, and steady formation of droplets (e.g., without jetting of fluid from holes of the membrane).

In variations, the hole diameter can range from 0.02 micrometers to 30 micrometers, and in examples, the holes can have an average hole diameter of 0.02 micrometers, 0.04 micrometers, 0.06 micrometers, 0.08 micrometers, 0.1 micrometers, 0.5 micrometers, 1 micrometers, 2 micrometers, 3 micrometers, 4 micrometers, 5 micrometers, 6 micrometers, 7 micrometers, 8 micrometers, 9 micrometers, 10 micrometers, 20 micrometers, 30 micrometers, any intermediate value, or greater than 30 micrometers (e.g., with use of membrane having a thickness greater than or otherwise contributing to a hole depth greater than 100 micrometers).

In variations, the hole depth can range from 1 micrometer to 200 micrometers (e.g., in relation to thickness of the membrane layer) or greater, and in examples the hole depth (e.g., as governed by membrane thickness) can be 1 micrometers, 5 micrometers, 10 micrometers, 20 micrometers, 30 micrometers, 40 micrometers, 50 micrometers, 60 micrometers, 70 micrometers, 80 micrometers, 90 micrometers, 100 micrometers, 125 micrometers, 150 micrometers, 175 micrometers, 200 micrometers, or any intermediate value.

In variations, the hole aspect ratio can range from 5:1 to 200:1, and in examples, the hole aspect ratio can be 5:1, 10:1, 20:1, 30:1, 40:1, 50:1, 60:1, 70:1, 80:1, 90:1, 100:1, 125:1, 150:1, 175:1, 200:1, or any intermediate value.

In variations, the hole-to-hole spacing can range from 5 micrometers to 200 micrometers or greater, and in examples, the hole-to-hole spacing is 5 micrometers, 10 micrometers, 20 micrometers, 30 micrometers, 40 micrometers, 50 micrometers, 60 micrometers, 70 micrometers, 80 micrometers, 90 micrometers, 100 micrometers, 125 micrometers, 150 micrometers, 175 micrometers, 200 micrometers, or greater. In a specific example, the hole-to-hole spacing is greater than 10 micrometers.

In examples, the hole orientation can be substantially vertical (e.g., during use in relation to a predominant gravitational force), otherwise aligned with a direction of applied force through the distribution of holes, or at another suitable angle relative to a reference plane of the membrane or other droplet generating substrate 120.

Additionally or alternatively, embodiments, variations, and examples of the methods described can be implemented by or by way of embodiments, variations, and examples of components described in U.S. application Ser. No. 17/687,080 filed 4 Mar. 2022, U.S. Pat. No. 11,242,558 granted 8 Feb. 2022, U.S. application Ser. No. 16/309,093 filed 25 May 2017, and PCT Application PCT/CN2019/093241 filed 27 Jun. 2019, each of which is herein incorporated in its entirety by this reference. In particular, methods described can be used to generate droplets of the set of droplets from the aqueous sample, where the aqueous droplets are each surrounded by a thin film of liquid that is immiscible with the aqueous droplets. The continuous phase of the emulsion is also aqueous, thereby producing a clear and gel-like emulsion, with variations of viscosity characteristics described. In relation to the reservoirs of the set of reservoirs 214, an aqueous sample can be loaded into a reservoir, and then a non-aqueous component (e.g., an oil) can be seated above the aqueous sample within the reservoir, prior to spinning the assembly comprising the first substrate 210 with the collecting containers 250 (e.g., under centrifugal force). The non-aqueous component can thus facilitate driving of the aqueous sample through the membrane, in order to eliminate sample dead volume (e.g., such that the volume of sample that is not dropletized is zero or negligible). In embodiments, the non-aqueous component is not driven through the membrane; however, in embodiments, the non-aqueous component may be driven through the membrane.

However, methods described can additionally or alternatively implement other system elements for sample reception and processing.

### 2.3 Method—Target-Specific Tagging and Amplification

Step S150 recites: performing target-specific tagging and amplification (and/or providing suitable environments for supporting such reactions), with the set of processing materials, for target regions associated with the set of targets. Step S150 preferably occurs within partitions generated from the sample, in digital format. However, Step S150 can be executed in another manner.

In one embodiment, the sample can be combined with an embodiment, variation, or example of the set of processing materials described above, and then partitioned such that template molecules of the sample occupy individual partitions with minimal overlap between different template molecules. Partitioning can be performed by passing the sample combined with the set of processing materials through a partitioning device (e.g., to generate droplets, to generate an emulsion with droplets provided in a continuous phase, etc.). Partitioning can alternatively be performed by distribution of the sample combined with the set of processing materials across a set of containers (e.g., microwells, nanowells, etc.). Partitioning can still alternatively be performed by distributing the sample combined with the set of processing materials across a substrate (e.g., as spots) and/or in another suitable manner.

A variation of Step S150 functions to denature the template material (e.g., DNA template) of the sample, anneal components to the target region(s) being evaluated, and amplify the target region(s) in a first stage, with generation of complements of allele-specific tail sequences in a second stage. Then, in subsequent stages of amplification, amounts of allele-specific tagged sequences increase in a manner that can be detected (e.g., through an optical-based detection method).

A first stage of sample processing can include denaturing of sample material (e.g., sample DNA) and processing the denatured sample material with primers (i.e., allele-specific forward primers grouped with corresponding reverse primers for each target). In the first stage, one of the allele-specific forward primers of the set of sample processing materials matches the target and, with the common reverse primer, amplifies the target region. As such, targets present in the sample are amplified upon interacting with respective target-specific forward primers.

A second stage of sample processing can include generation of target-specific sequences (e.g., tail sequences), where the common reverse primer binds to, elongates, and produces a complementary copy of a labeled tail sequence corresponding to the target.

A third stage of sample processing and subsequent stages can include one or more rounds of amplification/PCR to produce a detectable signal, whereby levels of tagged target-specific sequences increase until a detection threshold is reached and/or surpassed. In the third stage and subsequent stages of sample processing, labeled oligonucleotides bind to new complementary sequences (e.g., tail sequences), releasing fluorophores from corresponding quenchers to produce detectable signals for each target present in the sample. However, fluorophores corresponding to targets that are not present are not released and thus continue to be quenched during rounds of amplification. In particular, with regard to parameters associated with threshold cycles at which or beyond which amplified targets become detectable (e.g., Ct, Cp, Cq, etc.), step can include detecting and/or returning results indicative of target presence prior to the end-point of the process and/or at the end-point of the process (e.g., as in end-point PCR). Additionally or alternatively, real-time measurement of signals can be performed contemporaneously with each cycle of amplification.

### 2.4 Method—Signal Detection and Returned Outputs

As shown in FIG. 1A, the method 100 includes Step S110, which recites generating a count of a number of target molecules of a sample distributed across a set of partitions S110 (e.g., a high number of partitions at low occupancy, such as the configuration of partitions generated in Step S140); and returning a characterization of the sample based upon the count S120. In embodiments, the set of partitions includes a set of droplets of an emulsion stabilized in position (e.g., as a component of a clear emulsion having viscosity/gel-like characteristics described above) within a closed container, and generating the count comprises scanning a sheet and/or multiple sheets of droplets within the closed container using light sheet scanning or another suitable optical interrogation modality.

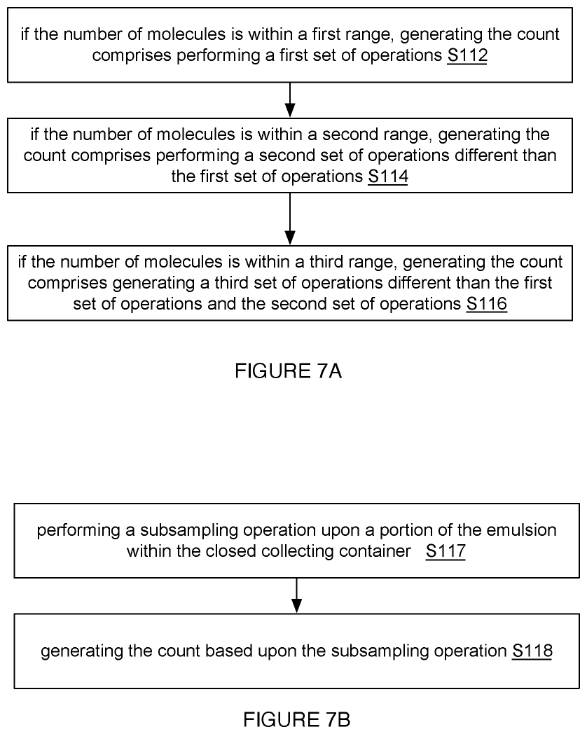

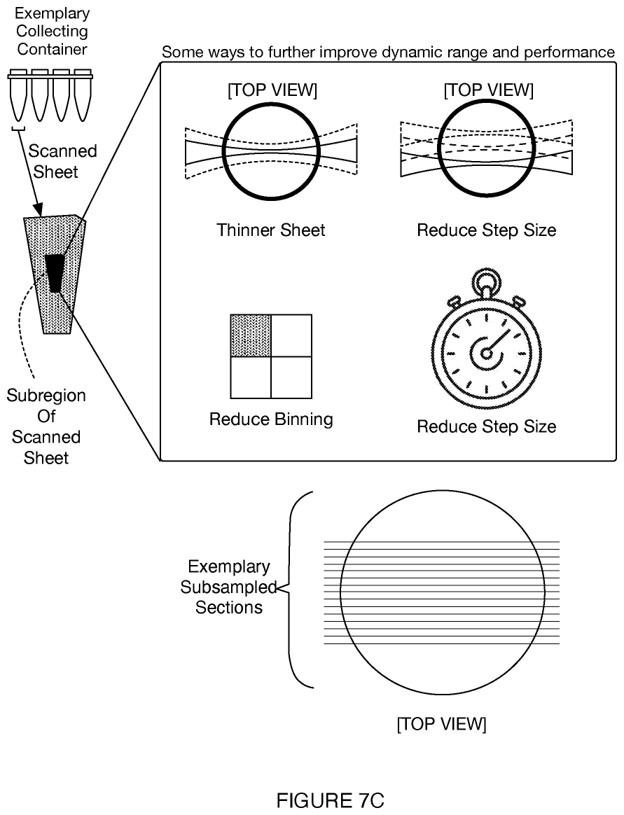

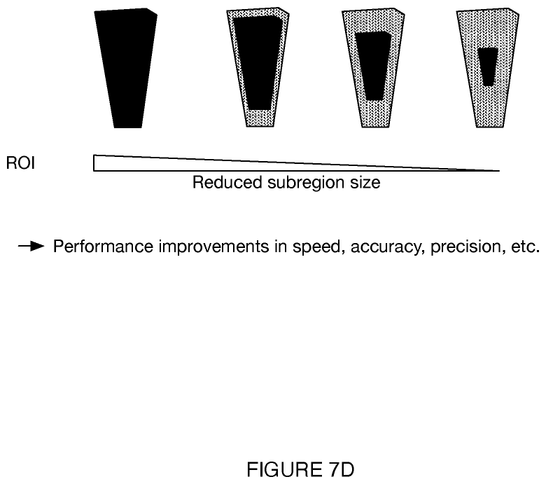

In relation to step S110, after readout data is generated (e.g., using lightsheet imaging, using another suitable imaging modality), generating the count can include transforming the readout data generated from frames of images, with indications of signal-positive partitions, into the count. With respect to transformation of the readout data, if the number of molecules is within a first range, generating the count comprises performing a first set of operations S112; if the number of molecules is within a second range, generating the count comprises performing a second set of operations different than the first set of operations S114; and if the number of molecules is within a third range, generating the count comprises generating a third set of operations different than the first set of operations and the second set of operations S116, as shown in FIG. 7A. While three ranges and three sets of operations are described, variations of the method can include generating the count for other numbers of ranges (e.g., one range, two ranges, four ranges, five ranges, six ranges, etc.) and/or with other numbers of sets of operations (e.g., one set of operations, two sets of operations, four sets of operations, five sets of operations, six sets of operations, etc.). In variations, for each target molecule being counted, operations for a first range of counts can be different for different target molecules.

In an example, the first range covers a range from a lower order of magnitude of 1 molecule to a higher order of magnitude of 10² molecules. In variations of the example, the first range can have a lower limit of an order of magnitude of 10⁰ targets or counts, 10¹ targets or counts, 10² targets or counts, 10³ targets or counts, or another suitable order of magnitude. In variations of the example, the first range can have an upper limit of an order of magnitude of 10¹ targets or counts, 10² targets or counts, 10³ targets or counts, 10⁴ targets or counts or another suitable order of magnitude.

In an example, the second range covers a range from a lower order of magnitude of 10² molecules to a higher order of magnitude of 10⁵ molecules. In variations of the example, the second range can have a lower limit of an order of magnitude of 10¹ targets or counts, 10² targets or counts, 10³ targets or counts, 10⁴ targets or counts, 10⁵ targets or counts, 10⁶ targets or counts, or another suitable order of magnitude. In variations of the example, the second range can have an upper limit of an order of magnitude of 10³ targets or counts, 10⁴ targets or counts, 10⁵ targets or counts, 10⁶ targets or counts, 10⁷ targets or counts or another suitable order of magnitude.

In an example, the third range covers a range from a lower order of magnitude of 10⁵ molecules to a higher order of magnitude of 10⁶ molecules. In variations of the example, the third range can have a lower limit of 10³ targets or counts, 10⁴ targets or counts, 10⁵ targets or counts, 10⁶ targets or counts, 10⁷ targets or counts, 10⁸ targets or counts, or another suitable order of magnitude. In variations of the example, the third range can have an upper limit of 10⁴ targets or counts, 10⁵ targets or counts, 10⁶ targets or counts, 10⁷ targets or counts, 10⁸ targets or counts, 10⁹ targets or counts, 10¹⁰ targets or counts, or another suitable order of magnitude.

The ranges can thus overlap or may alternatively not overlap. For instance, counts that fall within overlapping ranges of subportions of the dynamic range can be assessed using different sets of operations, and the higher quality count (e.g., in relation to confidence, in relation to accuracy, in relation to another quality metric) can be returned for characterization of the sample.

### 2.4.1 Counting Operations within Different Portions of the Entire Dynamic Range

In embodiments, generating the count in Step S110 can include enumerating positive droplets of the set of droplets upon scanning a set of sheets of droplets within the closed container using light sheet scanning S112, and enumerating positive droplets identified from the set of sheets S114. Relatedly, generating the count can include enumerating positive partitions (e.g., droplets) of the set of partitions, wherein a partition of the set of partitions is identified as a positive droplet based upon satisfaction of a set of criteria S116 including: a signal intensity criterion, an optical property criterion, a shape criterion, a morphology criterion, a spatial criterion, and chromaticity criterion from readout data acquired upon performing optical interrogation of the set of partitions, as described. Different criteria can thus reduce or eliminate false positives that could adversely affect counts, where false positives can be attributed to air bubbles, dust, edge fluorescence from damaged collecting containers, merged droplets, merged partitions, emulsion interface effects (e.g., due to presence of micelles, etc.), and/or other artifacts that could produce false positive counts. Different criteria can additionally or alternatively reduce or eliminate noise that could adversely affect counts, where noise can be attributed to reagents used (e.g., nuclease free water, Tris-EDTA buffer, oil phases, additives, imaging fluids, PCR master mix, etc.) according to methods described.

Signal Intensity Criterion: In variations, and with respect to a partition/droplet, the signal intensity criterion is evaluated for a set of pixels depicting the droplet, and the signal intensity criterion is based upon one or more of: a maximum signal intensity of the set of pixels, a maximum convoluted intensity of the set of pixels, a difference between the maximum intensity and the minimum intensity of the set of pixels, a total intensity of the set of pixels, a sum of signal intensities, a mean of signal intensities, a median of signal intensities, and another suitable representation of signal intensities.

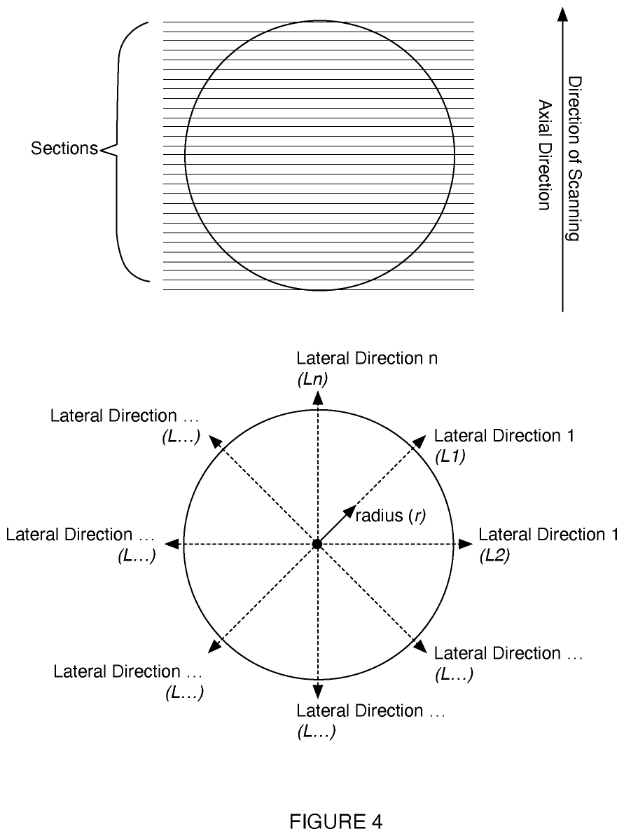

Optical Property Criterion: In variations, and with respect to a partition/droplet, the optical property criterion is evaluated for a set of pixels depicting the droplet, and the optical property criterion is based upon one or more of: a lateral intensity gradient across the set of pixels (across a direction of pixels in which a portion of the droplet/partition is depicted), an axial intensity gradient across the set of pixels (across a direction of pixels in which a portion of the droplet/partition is depicted), second order intensity gradients across the set of pixels along a set of directions (e.g., an axial direction and a lateral direction), and intensity uniformity across the set of pixels. Axial and lateral directions for a droplet/partition are shown in FIG. 4, with respect to a droplet. In relation to the lateral intensity gradient criterion, pixels along a set of lateral directions, as shown in FIG. 4 (where lateral directions are shown as L1, L2, . . . , Ln), are processed to determine an average lateral intensity gradient (e.g., in relation to droplet/partition radius).

Shape Criterion: In variations, and with respect to a partition/droplet, the shape criterion is evaluated for a set of pixels depicting the droplet, and the shape criterion is based upon one or more of: a droplet radius determined from the set of pixels prior to removing background pixels, a droplet radius determined from the set of pixels after removing background pixels, a droplet eccentricity determined from the set of pixels prior to removing background pixels, a droplet eccentricity determined from the set of pixels after removing background pixels, and a droplet radius of gyration.

Morphology Criterion: In variations, and with respect to a partition/droplet, the morphology criterion is evaluated for a set of pixels depicting a three-dimensional profile of a representative positive droplet determined from a set of control samples.

Chromaticity Criterion: In variations, and with respect to a partition/droplet, the chromaticity criterion covers combinations of color/label signals, evaluated for a set of pixels of a representative positive droplet determined from a set of control samples. In one example, if partitions with target molecules are in observed in a first channel, and a second reference channel, such partitions are filtered out as false positives.

Spatial Criterion: In variations, and with respect to a partition/droplet, the spatial criterion relates to location of droplets within space (e.g., within the container).

In relation to the set of pixels used to evaluate a droplet/partition with respect to different criteria described, the set of pixels can be acquired from image data corresponding to more than a single cross-section (e.g., multiple lightsheet image frames corresponding to multiple cross-sections). Alternatively, the set of pixels can be acquired from image data corresponding to a single cross-section (e.g., lightsheet image frame for a single cross-section).

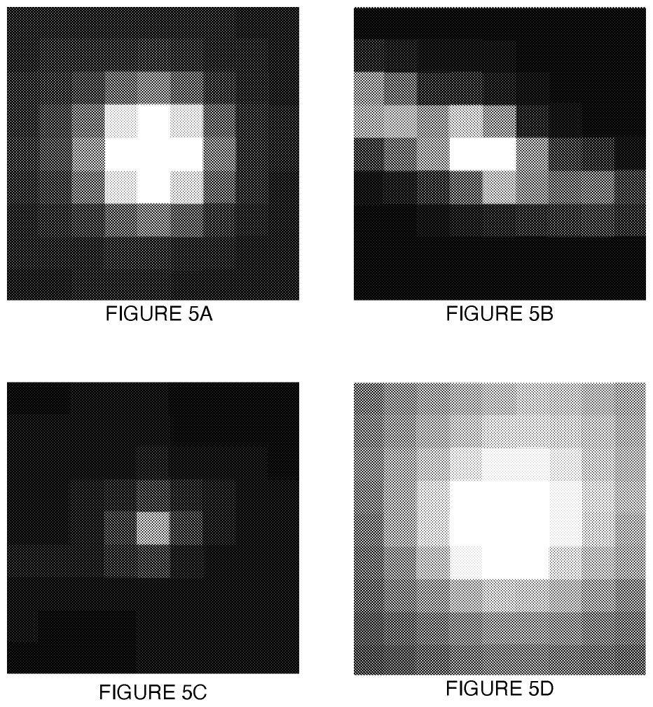

An example of a positive droplet is shown in FIG. 5A, and examples of negative droplets are shown in FIGS. 5B, 5C, and 5D. A stack of cross sectional images for each droplet and/or a projection of multiple cross sectional images for each droplet onto a two-dimensional space can also be used for control and/or training data.

With respect to increasing specificity of positive droplet identification from readout data generated from lightsheet images, generating the count in Step S110 can include generating the count upon processing input data produced from the readout data, with a machine learning model. The machine learning model can be trained with a training dataset where the training dataset is generated from processing of a set of control samples that include positive droplets (e.g., droplets individually containing a single target) and negative droplets (e.g., droplets that do not contain a single target). Increasing specificity of positive droplet identification produces high-confidence positive partition calling in rare-molecule samples (e.g., samples at lower order of magnitude ranges of the entire dynamic range).

The machine learning model can include classification model architecture, where the classification model architecture is trained with a supervised learning approach using labelled training data from control samples in order to allow the trained model to accurately identify positive droplets based upon a set of features. Features of pixels and/or partitions (e.g., droplets) of training data can be used to train the classification model, and features of pixels and/or partitions of new input test data (e.g., generated from subsequent scans of processed samples) can be used to generate counts of targets from such processed samples. As another approach, features can be learned from raw data instead of extracting known features, and/or features can be classified directly by feeding raw images (droplet candidates+surrounding pixels) into training models.

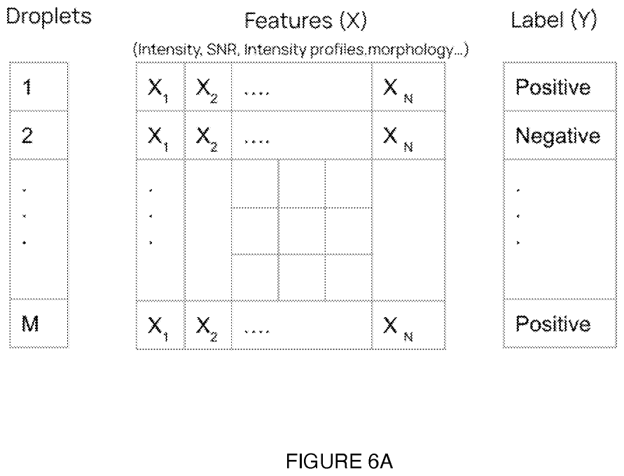

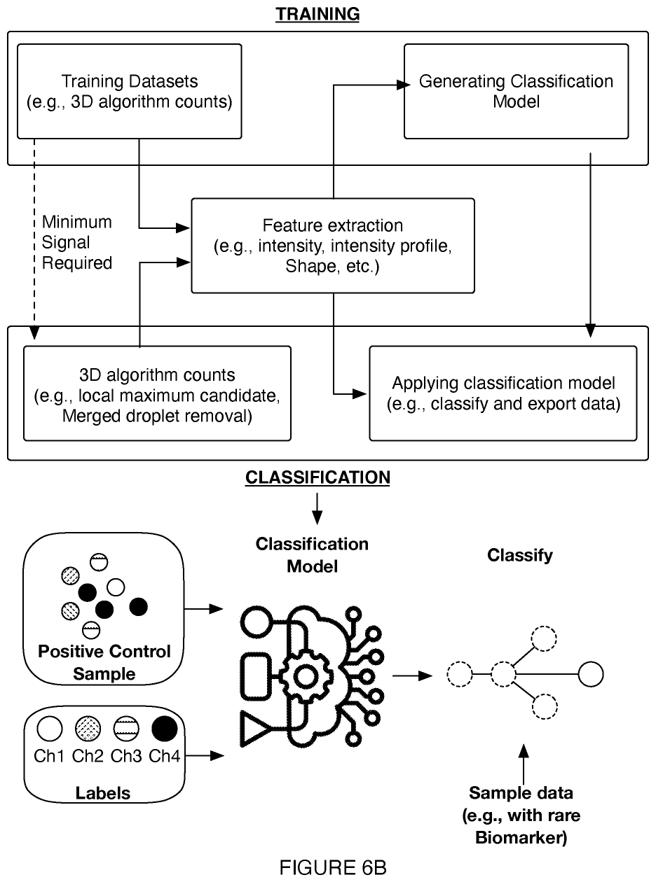

Exemplary droplet features applied by and/or used to train the classification model architecture can include one or more of: a droplet intensity feature, a signal to noise ratio, an intensity uniformity feature (e.g., determined from an integrated total of droplet pixel intensity divided by [droplet area multiplied by droplet intensity]), a first order intensity gradient in a polar/lateral direction (e.g., dI/dr), a second order intensity gradient in a polar/lateral direction (e.g., d2I/dr2), a first order intensity gradient in an axial direction (e.g., dI/dz), a second order intensity gradient in a polar direction (e.g., d2I/dz2), an integrated intensity (e.g., integrated intensity of all pixels representing a droplet), an eccentricity feature (e.g., eccentricity determined from a ratio of: [difference between a major droplet diameter and a minor droplet diameter]:[sum of a major droplet diameter and a minor droplet diameter]), a radius of gyration (e.g., determined from an square-root of integrated total of droplet radius squared multiplied by droplet pixel intensity, divided by integrated total droplet pixel intensity), and a droplet correlation with a three-dimensional (3D) profile of a positive droplet generated from a control sample. As such, a droplet can be labeled as a positive droplet (e.g., a droplet originally containing a single target) based upon a set of features, and the classification model can be trained to accurately label a test droplet at positive based upon the set of features (shown in FIG. 6A). An exemplary structure for training the classification model is shown in FIG. 6B.

In embodiments where there is spatial variation of droplet features associated with positive droplets and/or negative droplets at different positions within the closed collecting container, the classification model architecture can include subarchitecture for accurately labeling positive droplets based upon location (e.g., coordinates x, y, z) within the closed collecting container. Alternatively, in embodiments where there is spatial variation of droplet features associated with positive droplets and/or negative droplets at different positions within the closed collecting container, the classification model architecture can include subarchitecture for scaling features for determination of positive droplets, such that returning outputs of the classification model is based upon scaled feature values (e.g., effectively normalizing features across different droplet positions). While embodiments, variations, and examples of machine learning models (e.g., in relation to inputs, outputs, and training) are described above, models implemented for positive droplet identification can additionally or alternatively include other architecture components. For instance, statistical analyses and/or machine learning model architecture can be characterized by a learning style including any one or more of: supervised learning, unsupervised learning, semi-supervised learning, reinforcement learning (e.g., using a Q-learning algorithm, using temporal difference learning, etc.), and any other suitable learning style. Furthermore, any machine learning algorithms can implement any one or more of: a classification algorithm (e.g., a decision tree learning method such as classification and regression tree, chi-squared approach, random forest approach, multivariate adaptive approach, gradient boosting machine approach, etc.), a regression algorithm, an instance-based method, a regularization method, a Bayesian method, a kernel-based approach (e.g., a support vector machine, a linear discriminate analysis, etc.), a clustering method (e.g., k-means clustering), an associated rule learning algorithm (e.g., an Apriori algorithm), an artificial neural network model, a deep learning algorithm, a self-attention model, a dimensionality reduction method (e.g., principal component analysis, partial least squares regression, etc.), an ensemble method, and any suitable form of algorithm.

With respect to generating the count at higher order of magnitude ranges of the entire dynamic range (e.g., for counts having an order of magnitude greater than 10² counts), Step S110 can include modified steps that improve counting performance, with respect to reducing computational load of counting, with respect to improving the speed at which a count can be generated post-generation of readout data, with respect to reducing scanning time for each channel of a set of channels used to interrogate partitions of the sample, and/or in other manners.

In one variation, generating the count S110 can include: performing a subsampling operation upon a portion of the emulsion within the closed collecting container S117, and generating the count based upon the subsampling operation S118, as shown in FIG. 7B.

In embodiments where the closed container comprises a width and a lateral axis referenced to a center of the closed container, performing the subsampling operation in Step S117 and generating the count in Step S118 can include the following: omitting processing of a subset of sheets of image data (e.g., lightsheet image data) positioned away from the center of the closed container along the lateral axis. In particular, image data/readout data can be generated for a subset of cross-sections of the closed collecting container containing the emulsion, where the subset of sheets can be positioned closer to the center of the tube (e.g., within a distance from the center of the tube less than 90% of the radius of the tube, less than 80% of the radius of the tube, less than 70% of the radius of the tube, less than 60% of the radius of the tube, less than 55% of the radius of the tube, less than 50% of the radius of the tube, less than 45% of the radius of the tube, less than 40% of the radius of the tube, less than 35% of the radius of the tube, less than 30% of the radius of the tube, less than 25% of the radius of the tube, less than 20% of the radius of the tube, less than 15% of the radius of the tube, less than 10% of the radius of the tube, etc.). Performing the subsampling operation in Step S117 can include generating a partial count based upon the subset of sheets, where the partial count is based upon a positive droplet density per unit area or volume captured in the subset of sheets, and generating the count in Step S118 can include extrapolating the partial count to a total count (e.g., based upon the droplet density and the total volume of the emulsion within the closed collecting container). An example of subsampled sheets for the subsampling operation is shown in FIG. 7C. In some instances, performing the subsampling operation in Step S117 and generating the count in Step S118 improves counting percent coefficient of variation, thus improving counting precision (e.g., in relation to expected counts vs. counts determined using Steps S117 and S118). In some instances, performing the subsampling operation in Step S117 and generating the count in Step S118 improves counting accuracy as well (e.g., in relation to expected counts vs. counts determined using Steps S117 and S118), due to higher likelihood of false positive droplets near edges/walls of the closed collecting container and other interfaces between the emulsion and the closed collecting container.

In variations, a referenced center of the closed collecting container may not necessarily geometrical center or center of volume. The center of the closed collecting container is determined based on the distance from the edges of the closed collecting container (e.g., distance from a top interface within the closed collecting container, and distance from back of the closed collecting container.

In embodiments where the emulsion comprises an upper surface within the closed container, performing the subsampling operation in Step S117 and generating the count in Step S118 can include the following: omitting processing of regions of sheets (e.g., cross section images) proximal to the upper surface and proximal to an internal boundary of the closed container. An example of a subsampled region of a sheet for the subsampling operation is shown in FIGS. 7C and 7D.

Generating the count in Step S110 (e.g., using steps S117, S118, etc.) can include generating the count for a sample within 120 seconds, within 100 seconds, within 90 seconds, within 80 seconds, within 70 seconds, within 60 seconds, within 50 seconds, within 40 seconds, within 30 seconds, 20 seconds, within 15 seconds, within 10 seconds, within 9 seconds, within 8 seconds, within 7 seconds, within 6 seconds, within 5 seconds, within 4 seconds, within 3 seconds, within 2 seconds, within 1 second, within 0.5 seconds, within 0.1 seconds, or less.

In examples, generating the count can include enumerating positive droplets of the set of droplets upon scanning a set of sheets of droplets spanning less than 25% of an internal volume of the closed container using light sheet scanning, and enumerating positive droplets identified from the set of sheets. However, in variations, generating the count can include scanning a set of sheets of droplets spanning less than 80% of an internal volume of the closed container, less than 70% of an internal volume of the closed container, less than 60% of an internal volume of the closed container, less than 50% of an internal volume of the closed container, less than 40% of an internal volume of the closed container, less than 30% of an internal volume of the closed container, less than 20% of an internal volume of the closed container, less than 10% of an internal volume of the closed container, or smaller.

In variations, generating the count in Step S110 can include projecting candidate positive droplets, generated from processing input data from scanning the closed collecting container with the classification model, onto a mapping (e.g., two dimensional mapping (UMAP)) of droplets, and counting only droplets of a cluster of positive droplets from the two dimensional mapping of droplets.

In embodiments, increasing the dynamic range of counting can involve one or more of: reducing a thickness of a lightsheet used to scan the closed container, reducing a step size distance of scanning along a scanning direction through the closed container, reducing binning, increasing magnification (e.g., by way of instrumentation) for observing partitions, using computational methods (e.g. deconvolution) to increase axial resolution of image data of partitions, and/or increasing lightsheet exposure time (shown in FIG. 7C).

### 2.4.2 Signal Detection from Optical Interrogation and Detection of Partition Signals

In variations, detection of signals can include irradiating processed sample material with suitable excitation wavelengths of light, and/or receiving emitted wavelengths of light corresponding to released dyes/fluorophores. As such detection of signals can be implemented by an optical signal detection subsystem (e.g., imaging subsystem), including embodiments, variations, and examples of systems described above. In particular, detection subsystems can be structured for detection of signals from partitions (e.g., by light sheet imaging, by fluorescence microscopy, by confocal microscopy, by 3D holographic microscopy, by 3D tomographic microscopy, by another suitable optical detection subsystem, etc.) using combinations of filters and/or color channels, where signals from individual partitions are detected in a high-partition number but low-occupancy regime. As such, detection can be performed for partitions arranged in 3D (e.g., as in droplets of an emulsion within a container), in 2D (e.g., for a monolayer or bi-layer of partitions at a substrate), and/or in another suitable format.

In variations, the sample can be processed with the set of processing materials in coordination with distribution of the sample across a set of partitions, where the set of partitions can include droplets (e.g., droplets of an emulsion, droplets provided in a sheathing fluid, gel droplets, other forms of droplets), microchambers, microwells, spotted samples on a substrate, and/or other partitions. As such, the partitions can be provided within one or more of: a container configured for centrifugation (e.g., a centrifuge tube, a microcentrifuge tube, etc.), a process container for PCR (e.g., a PCR tube), a strip tube, a plate having wells (e.g., a microtiter plate, a multi-well plate, a microwell plate, a nanowell plate, etc.), or another suitable collecting container. Additionally or alternatively, partitions can include regions of sample provided in another manner upon a substrate (e.g., spotted onto a substrate/slide).

With respect to sample processing using the set of processing materials, reactions within individual partitions can thus produce signals that are detected by systems that can detect signals from multiple partitions or all partitions simultaneously in a distinguishable manner (e.g., with a 3D scanning technique, as described above). Alternatively, reactions within individual partitions can produce signals that are detected by systems that can detect signals from individual partitions in a sequential manner.

Characterizing a set of targets of a sample (e.g., in relation to presence/absence, in relation to counts of targets), upon scanning the set of partitions, can include: generating a multivariable vector of emission values (e.g., emitted intensity values across the set of available color channels), for each detected emitted signal from a respective partition, paired with the excitation parameters used to excite the set of partitions; performing a clustering operation with the multivariable vectors of emission values generated from the set of partitions (e.g., where clustering can be performed sequentially/hierarchically, with a subset of variables first, and then another subset, etc.); sorting partitions of the set of partitions into a set of categories corresponding to targets of the set of targets, based upon the clustering operation and a known set of probes used to tag the set of targets; and generating a count of each of the set of targets represented in the set of partitions, based upon said sorting. In variations, the clustering operation can include performance of a co-localization operation, whereby scanning deviations are corrected for in order to further delineate/provide stronger discrimination between different clusters of partitions. Additionally or alternatively, dimension-reduction algorithms can further include one or more of: principal component analysis (PCA), k-means clustering, t-stochastic neighbor embedding (t-SNE), UMAP, and/or other dimension-reduction algorithms. Examples of clustering algorithms can be: k-means, DBSCAN, or more generally, hierarchical-based, density-based, centroid based, etc. and/or other algorithms. In variations, characterizing partitions can further include identifying partitions that are signal positive in more than one channel, in relation to color combinatorics described above.

### 2.4.3 Characterization the Sample based on the Count

Step S120 recites: returning a characterization of the sample based upon the count S120. Step S120 functions to provide information pertaining to presence or absence of the set of targets associated with the sample being evaluated, and/or presence or absence of variants of the set of targets. The characterization can then be used to provide diagnostics and/or to support diagnostics of the organism(s) from which the processed sample was sourced, and/or to provide quality for conclusiveness of diagnostic results. Additionally or alternatively, the characterization can be used to guide provision of therapeutics (e.g., personalized therapeutics) corresponding to determined states of the organism(s) from which the processed sample was sourced, in order to improve or maintain health statuses of the organism(s).

In specific applications, the characterization can be used to inform diagnostics, provide other characterizations (e.g., of disease resistance, of disease predisposition, of genetic relationships, etc.) and/or guide generation of therapeutics associated with non-invasive prenatal testing (described above and in more detail below). More broadly, outputs of step S120 can be used to characterize (e.g., based on relative abundance measurements) self genetic material (e.g., genetic material of an organism) and non-self genetic material (e.g., genetic material not of the organism, genetic material of a different organism) from a sample.

Additionally or alternatively, in specific applications, the characterization can be used to inform characterizations of a subject from which the sample is sourced, in relation to one or more of: cancers, integumentary system conditions, skeletal system conditions, muscular system conditions, lymphatic system conditions, respiratory system conditions, digestive system conditions, nervous system conditions, endocrine system conditions, cardiovascular system conditions, urinary system conditions, reproductive system conditions, and/or other conditions.

Outputs can additionally or alternatively support at least one of: pathogen detection, non-invasive prenatal testing, organ transplantation analysis, forensics, and oncology, based upon the quantitative analysis.

In other specific applications, the characterization can be used to inform diagnostics, provide other characterizations (e.g., of disease resistance, of disease predisposition, of genetic relationships, etc.) and/or guide generation of therapeutics in the context of other multicellular organisms, plants, fungi, unicellular organisms, viruses, and/or other subjects.

In another specific application area, the characterization can be used to support or inform diagnostic assays for NIPT, other prenatal tests, and other sample characterization techniques. In specific examples, aspects of the present disclosure can be used to detect various trisomies and/or other aneuploidies in a multiplexed manner. In examples, the compositions, methods, and systems can involve testing for aneuploidies in chromosome 13, chromosome 18, and/or chromosome 21. In other examples, the compositions, methods, and systems can involve testing or characterization of aneuploidies or other genetic disorders in other chromosomes. In specific examples, aspects of the present disclosure can be used to target genomic diseases, associated with but not limited to one or more locis associated with: chromosome 21, chromosome 18, chromosome 13, chromosome X, chromosome Y, 22q11.2 deletion/DiGeorge's Syndrome, Down syndrome, Klinefelter syndrome, XYY syndrome, Turner syndrome, deletion syndromes, other chromosomal abnormalities, rare mutation detection, minimal residual disease, and/or other diseases.

Relatedly, the characterization can be used to generate chromosomal counts and differential chromosomal count ratios across different fetal fraction scenarios. In particular, due to the relatively low fetal fraction in maternal cell free DNA, a higher order level of DNA counting is required for accurate determination and in order to achieve suitable statistical confidence to distinguish between non-aneuploid and aneuploid fetuses. Current approaches for NIPT rely on platforms such as next generation sequencing (NGS) and microarrays, which are expensive with complex multi-day workflows, limiting its deployment in typical hospital laboratories. On the other hand, platforms such as digital PCR, while being a gold standard analytical platform, is at least an order of magnitude away in relation to generating levels of count suitable for diagnosis. Furthermore, other digital PCR platforms suffer from low precision due low partitioning capabilities, and rely upon Poisson correction factors. Aspects of the present disclosure include digital assay technologies that far exceed the precision of standard digital PCR platforms, and can perform at a DNA counting range akin to NGS, which makes digital ultraPCR suitable for NIPT. Example results include production of high counts (e.g., from a 10 mL sample, from a smaller than 10 mL sample, from a larger than 10 mL sample) required for NIPT fetal aneuploidy screening.

In examples, the characterization can achieve high performance counting of greater than n counts, with partitioning performed in a manner such that that the occupancy per template remains in the single molecule regime. Thus, there is minimal or no overlap between different template molecules with individual partitions and no statistical correction is needed (e.g., due to non-existent partitioning error). This allows the systems, methods, and compositions to enable measurement performance down to at least a 2% difference in counts (e.g., where a 2% difference in counts is equivalent to a 4% fetal fraction from fetus with trisomy or monosomy). In examples, n 50,000 counts per chromosome for each of a set of chromosomes of interest, 60,000 counts per chromosome for each of a set of chromosomes of interest, 70,000 counts per chromosome for each of a set of chromosomes of interest, 80,000 counts per chromosome for each of a set of chromosomes of interest, 90,000 counts per chromosome for each of a set of chromosomes of interest, 100,000 counts per chromosome for each of a set of chromosomes of interest, 120,000 counts per chromosome for each of a set of chromosomes of interest, 130,000 counts per chromosome for each of a set of chromosomes of interest, 140,000 counts per chromosome for each of a set of chromosomes of interest, 150,000 counts per chromosome for each of a set of chromosomes of interest, 160,000 counts per chromosome for each of a set of chromosomes of interest, 170,000 counts per chromosome for each of a set of chromosomes of interest, 180,000 counts per chromosome for each of a set of chromosomes of interest, 190,000 counts per chromosome for each of a set of chromosomes of interest, 200,000 counts per chromosome for each of a set of chromosomes of interest, 210,000 counts per chromosome for each of a set of chromosomes of interest, 220,000 counts per chromosome for each of a set of chromosomes of interest, 230,000 counts per chromosome for each of a set of chromosomes of interest, 240,000 counts per chromosome for each of a set of chromosomes of interest, 250,000 counts per chromosome for each of a set of chromosomes of interest, 260,000 counts per chromosome for each of a set of chromosomes of interest, 270,000 counts per chromosome for each of a set of chromosomes of interest, 280,000 counts per chromosome for each of a set of chromosomes of interest, 290,000 counts per chromosome for each of a set of chromosomes of interest, 300,000 counts per chromosome for each of a set of chromosomes of interest, or even other counts per chromosome for each of a set of chromosomes of interest.

Embodiments of the methods described can be further adapted for other applications of use.

### 3. Computer Systems

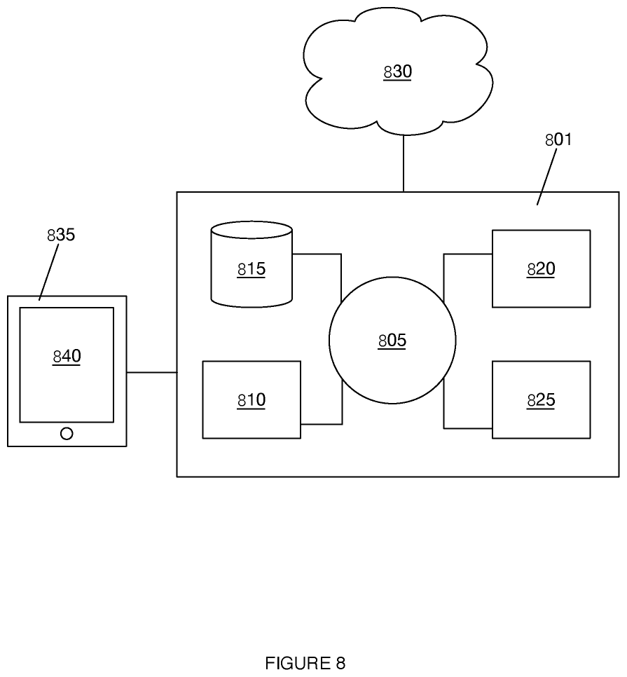

The present disclosure provides computer systems that are programmed to implement methods of the disclosure. FIG. 8 shows a computer system 801 that is programmed or otherwise configured to, for example, generate a count of a number of targets within a sample, according to the performance specification described. Performing the digital analysis can include: generating a count of a number of target molecules of a sample distributed across a set of partitions (e.g., a high number of partitions at low occupancy); and returning a characterization of the sample based upon the count.

The computer system 801 can additionally or alternatively perform other aspects of digital assays for characterizations involving other loci of interest, with applications of use described above.

The computer system 801 can regulate various aspects of analysis, calculation, and generation of the present disclosure, such as, for example, generating a plurality of partitions (e.g., from an aqueous mixture including sample material and materials for an amplification reaction) within a collecting container at a desired rate, transmitting heat to and from the plurality of partitions within the collecting container, performing an optical interrogation operation with the plurality of partitions within the collecting container, and/or performing one or more digital multiplexed assay steps. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet.

In some embodiments, the network 830 is a telecommunication and/or data network. The network 830 can include one or more computer servers, which can enable distributed computing, such as cloud computing. For example, one or more computer servers may enable cloud computing over the network 830 (“the cloud”) to perform various aspects of analysis, calculation, and generation of the present disclosure, such as, for example, generating a plurality of droplets within a collecting container at a predetermined rate or variation in polydispersity. Such cloud computing may be provided by cloud computing platforms such as, for example, Amazon Web Services (AWS), Microsoft Azure, Google Cloud Platform, and IBM cloud. In some embodiments, the network 830, with the aid of the computer system 801, can implement a peer-to-peer network, which may enable devices coupled to the computer system 801 to behave as a client or a server.

The CPU 805 may comprise one or more computer processors and/or one or more graphics processing units (GPUs). The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some embodiments, the circuit is an application specific integrated circuit (ASIC).

The storage unit 815 can store files, such as drivers, libraries and saved programs. The storage unit 815 can store user data, e.g., user preferences and user programs. In some embodiments, the computer system 801 can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some embodiments, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Embodiments of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, a visual display indicative of stages of or results from performing a digital analysis of a sample distributed across a set of partitions. The UI 840 can additionally or alternatively be adapted for performing other digital assays involving other loci of interests and/or other calculations, as described. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, generate a plurality of droplets within a collecting container with desired characteristics.

### 4. Conclusion

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

As a person skilled in the art will recognize from the previous detailed description and from the figures and claims, modifications and changes can be made to the preferred embodiments of the invention without departing from the scope of this invention defined in the following claims.

What is claimed is:

1. A method comprising:
(a) providing a plurality of partitions, wherein the plurality of partitions is within a continuous phase within a container;
(b) using an imaging system to scan the plurality of partitions across a set of optical channels to detect signals from at least a subset of the plurality of partitions; and
(c) using the signals detected in (b) to generate a count of a number of the target molecules.

2. The method of claim 1, wherein the target molecules comprise nucleic acid molecules.

3. The method of claim 1, wherein the plurality of partitions comprises a set of wells.

4. The method of claim 1, wherein (b) comprises scanning a sheet of the container using a light sheet imaging system.

5. The method of claim 1, wherein the plurality of partitions comprises at least 1 million partitions.

6. The method of claim 1, wherein the plurality of partitions comprises at least 5 million partitions.

7. The method of claim 1, wherein (c) is performed within 5 seconds.

8. The method of claim 1, wherein the plurality of partitions comprises a plurality of nucleic acid molecules.

9. The method of claim 8, further comprising before (b), amplifying the plurality of nucleic acid molecules.

10. The method of claim 1, wherein (c) comprises identifying partitions of the set of partitions comprising the target molecule based on satisfaction of an optical property criterion, a shape criterion, and a morphology criterion.

11. The method of claim 10, wherein the optical property criterion is based on a lateral or axial intensity gradient across a set of pixels.

12. The method of claim 10, wherein the shape criterion is based on a radius of a partition of the set of partitions.

13. The method of claim 10, wherein the morphology criterion is based on a three-dimensional profile of a partition profile of a control sample.

14. The method of claim 10, wherein:
i) wherein the optical property criterion is based on a lateral or axial intensity gradient across a set of pixels;
ii) wherein the shape criterion is based on a radius of a partition of the set of partitions; and
iii) wherein the morphology criterion is based on a three-dimensional profile of a partition profile of a control sample.

15. The method of claim 1, wherein the count of the number of target molecules is between 1 target molecule and 1,000,000 target molecules.

16. The method of claim 10, wherein identifying partitions of the set of partitions comprising the target molecule is further based on satisfaction of a signal intensity criterion.

17. The method of claim 16, wherein the signal intensity criterion is based upon maximum convoluted intensity of the set of pixels, a difference between the maximum intensity and the minimum intensity of the set of pixels, and a total intensity of the set of pixels.

18. The method of claim 16, wherein the signal intensity criterion is based upon a maximum signal intensity of the set of pixels.

19. The method of claim 1, wherein the container has a volumetric capacity of 50 microliters.

20. The method of claim 1, wherein the container is a tube.
